(19) United States
(12) Patent Application Publication
(10) Pub. No.: US 2012/0201749 A1
(43) Pub. Date: Aug. 9, 2012

(54) NP-1 Antagonists and Their Therapeutic Use

(76) Inventors: Melanie Jayne Crawshaw, Cambridge (GB); Philip Spencer Fallon, Cambridge (GB); Ashley Nicholas Jarvis, Cambridge (GB); Jonathan Raymond Powell, Cambridge (GB); Natalie Louise Winfield, Cambridge (GB); David Selwood, London (GB); Ian Charles Zachary, London (GB); Haiyan Jia, London (GB)

(21) Appl. No.: 13/391,686

(22) PCT Filed: Aug. 25, 2010
(86) PCT No.: PCT/GB2010/051413

§ 371 Date: Apr. 23, 2012

(30) Foreign Application Priority Data
Aug. 25, 2009 (GB) 0914856.0

(51) Int. Cl. A61K 31/4535; A61K 31/496; A61K 31/4436; C07D 413/14; A61K 49/10; A61P 25/28; A61P 35/00; A61P 37/00; A61K 51/00; C07D 409/14; A61K 31/5377

(52) U.S. Cl. 424/1.11; 546/196; 514/320; 544/364; 514/253.11; 546/281.4; 514/337; 544/130; 514/233.5; 424/9.3

(57) ABSTRACT

Compounds of formula (I): or a pharmaceutically acceptable salt thereof, wherein: W is arylene, heteroarylene or formula (a) each L is independently alkylene, alkenylene, alkynylene, a direct bond, arylene, cycloalkylene, alkylene-arylene, alkylene-C═O or —C═O; each X is independently an N-containing heteroarylene, N-containing cycloalkylene or NR; Y is N-containing heteroaryl, N-containing cycloalkyl, NR₂, OR¹, CN or CO₂R; Z₁ is formula (b); are useful in therapy, particularly in the therapy of neurodegeneration and cancer.

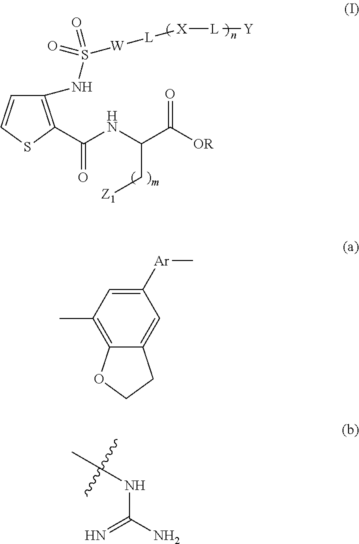

### FIELD OF THE INVENTION

This invention relates to compounds, which have NP-1 antagonist activity, and are therefore useful in therapy.

### BACKGROUND OF THE INVENTION

A non-tyrosine kinase transmembrane protein, neuropilin-1 (NP-1) is a receptor for members of the VEGF family of angiogenic cytokines, particularly VEGF-A₁₆₅, essential for vascular development, as well as a receptor for a family of molecules called semaphorins or collapsins which play a key role in the guidance of neuronal axons during mammalian development. In particular, NP-1 is known to mediate the growth cone-collapsing and chemorepulsive activity of semaphorin 3A. NP-1 has been shown to play a role in the primary T-cell immune response and in cellular entry of and infection by the Human T-cell Lymphotropic Virus, HTLV-1.

There are a number of conditions in which NP-1 may have a significant role in pathology. Such conditions include stroke, ischaemic eye disease, cancer, in particular lung cancer, and rheumatoid arthritis.

### SUMMARY OF THE INVENTION

New compounds have been discovered, which have surprisingly potent activity in antagonising VEGF binding to NP-1.

According to a first aspect, the present invention is a compound of formula I:

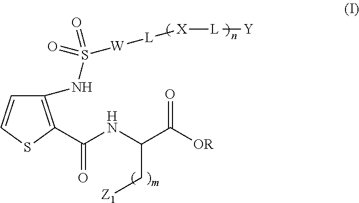

or a pharmaceutically acceptable salt thereof,

wherein:

W is arylene, heteroarylene or

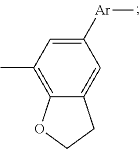

each L is independently alkylene, alkenylene, alkynylene, a direct bond, arylene, cycloalkylene, alkylene-arylene, alkylene-C═O or —C═O;

each X is independently an N-containing heteroarylene, N-containing cycloalkylene or NR;

Y is N-containing heteroaryl, N-containing cycloalkyl, NR₂, OR¹, CN or CO₂R;

Z₁ is

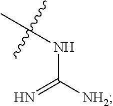

R is H or C₁-C₆ alkyl;

R¹ is H, C₁-C₆ alkyl or an amino acid;

n is 2, 3, 4 or 5; and

m is 1, 2 or 3.

According to a second aspect, the present invention is a compound according to formula II:

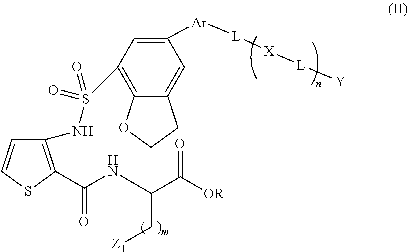

or a pharmaceutically acceptable salt thereof,

wherein:

each L is independently alkylene, alkenylene, alkynylene, a direct bond, arylene, cycloalkylene, alkylene-arylene, or alkylene-C═O;

each X is independently an N-containing heteroarylene, N-containing cycloalkylene or NR;

Y is N-containing heteroaryl, N-containing cycloalkyl, NR, OR¹, CN or CO₂R;

Z₁ is

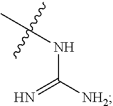

R is H or C₁-C₆ alkyl;

R¹ is H, C₁-C₆ alkyl or an amino acid;

n is 0, 1, 2, 3, 4 or 5; and

m is 1, 2 or 3.

### DESCRIPTION OF THE FIGURE

FIG. 1 is a graph showing the effects of a compound of the invention, compound 58 on tumour growth.

### DESCRIPTION OF PREFERRED EMBODIMENTS

It will be appreciated that the compounds according to the invention contain an asymmetrically substituted carbon atom. Specifically, there is a chiral centre in general formula I and II, where the arginine side-chain attaches to the main back-bone. The chiral configuration can either be R or S. Both enantiomers are included within the scope of the invention.

The presence of this asymmetric centre in the compounds of the invention can give rise to stereoisomers, and in each case the invention is to be understood to extend to all such stereoisomers, including enantiomers and diastereomers, and mixtures including racemic and non-racemic mixtures thereof.

It will also be appreciated that tautomers of the specific compounds of the invention exist, and these are included within the scope of the invention. These tautomers may be formed after the formal migration of a hydrogen atom, and the switch of a single bond and an adjacent double bond. Methods of tautomerization will be well known to those skilled in the art.

For the avoidance of doubt, when n is greater than 1, each of the X and each of the L groups in parenthesis, are selected independently. For example, where n is 2, i.e. (XL)-(XL), each X group may be different from the other one, and each L group may be different from the other one.

For the avoidance of doubt, the term if the situation exists where L is a direct bond, for example W-L-X, then that means that the L group is “absent”. In other words, using the example W-L-X, if L is a direct bond, then the W atom is directly attached to the X atom.

As used herein the terms “alkyl” or “alkylene” refer to a mono- or di-valent straight or branched-chain alkyl moiety, including for example, methyl, ethyl, propylene, isopropyl, butyl, tert-butyl, pentylene, hexyl and the like. Preferably, alkyl and alkylene groups each contains from 1 to 10 carbon atoms, respectively. More preferably, alkyl and alkylene means C₁-C₆ alkyl and C₁-C₆ alkylene, respectively.

As used herein, alkenyl preferably means a C₂-C₁₀ alkenyl group. Preferably, it is a C₂-C₆ alkenyl group. More preferably, it is a C₂-C₄ alkenyl group. The alkenyl radicals may be mono- or di-saturated, more preferably monosaturated. Examples include vinyl, allyl, 1-propenyl, isopropenyl and 1-butenyl. It may be divalent, e.g. propenylene

As used herein, alkynyl is preferably a C₂-C₁₀ alkynyl group which can be linear or branched. Preferably, it is a C₂-C₄ alkynyl group or moiety. It may be divalent.

Each of the alkyl, C₂-C₁₀ alkenyl and C₂-C₁₀ alkynyl groups may be optionally substituted with each other, i.e. C₁-C₁₀ alkyl optionally substituted with C₂-C₁₀ alkenyl. They may also be optionally substituted with aryl, cycloalkyl (preferably C₃-C₁₀), aryl or heteroaryl.

The terms “aryl” or “arylene” or “Ar” mean mono- or di-valent aromatic hydrocarbon moiety, and include phenylene, biphenyl or naphthyl group. The ring may be substituted by up to 5 substituents. Other possible substituents are C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl)amino C₁-C₃ alkyl, bis(C₁-C₃ alkyl)amino C₁-C₃ alkyl, C₁-C₃-acylamino, C₁-C₃ alkyl sulfonylamino, halo, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl. In a preferred embodiment, Ar is benzyl or benzylene.

The aryl or arylene ring is preferably 5 or 6-membered.

The terms “heteroaryl” or “heteroarylene” refer to mono-valent or di-valent aromatic ring systems, from which at least one ring atom is selected from, O, N, or S and includes for example benzofused furanyl, thiophenylene, thiophenylene (phenyl), pyridyl, indolyl, pyridazinyl, piperazinyl, pyrimidinyl, thiazolylene and the like. The heteroaryl or heteroarylene is preferably 5, 6 or 7-membered, and may be substituted by up to 5 substituents, for example by an amino, alkyl or carboxylic acid group, or the like. Other possible substituents are as listed above for “aryl” groups.

As used herein, cycloalkyl or cycloalkylene means a mono- or di-valent saturated ring system, which may contain heteroatoms such as N, O or S. An “N-containing cycloalkyl” must contain at least one N atom. Preferably, it contains two N atoms. Preferably, the ring contains 5 or 6 atoms. Examples are cyclohexyl or cyclopentylene. The ring may be substituted, preferably by at least one of the groups listed as possible substituents in the definition of “aryl”, above.

As used herein, heterocycle is a mono- or di-valent carbocyclic radical containing up to 4 heteroatoms independently selected from oxygen, nitrogen and sulphur.

The heterocyclic ring may be mono- or di-saturated. The radical may be optionally substituted with up to three substituents independently selected from C₁-C₆ alkyl, hydroxy, C₁-C₃ hydroxyalkyl, C₁-C₃ alkoxy, C₁-C₃ haloalkoxy, amino, C₁-C₃ mono alkylamino, C₁-C₃ bis alkylamino, C₁-C₃ acylamino, C₁-C₃ aminoalkyl, mono (C₁-C₃ alkyl)amino C₁-C₃ alkyl, bis(C₁-C₃ alkyl)amino C₁-C₃ alkyl, C₁-C₃-acylamino, C₁-C₃ alkyl sulfonylamino, halo e.g. F, nitro, cyano, trifluoromethyl, carboxy, C₁-C₃ alkoxycarbonyl, aminocarbonyl, mono C₁-C₃ alkyl aminocarbonyl, bis C₁-C₃ alkyl aminocarbonyl, —SO₃H, C₁-C₃ alkylsulfonyl, aminosulfonyl, mono C₁-C₃ alkyl aminosulfonyl and bis C₁-C₃-alkyl aminosulfonyl.

As used herein, the above groups can be followed by the suffix -ene. This means that the group is divalent, i.e. a linker group.

Preferably, at least one L is alkylene. Preferably, it is CH₂. More preferably, at least one L is a bond. Still more preferably, at least one L is arylene.

Preferably, W is benzylene.

Preferably X is NR, wherein R is as defined above. More preferably, X is a 6-membered cycloalkylene containing at least one N atom.

Preferably, Y is a 6-membered cycloalkyl containing at least one N atom. More preferably, Y is a substituted or unsubstituted 5-membered heteroaryl containing at least one N atom and one other atom selected from O or S and N. Still more preferably, Y is pyridine or Y is C₆H₄CN.

Preferably, n in structure II is 1 to 5. Preferably, n is structures I and II is 2. More preferably, n is 3.

Preferably Ar in structures I and II is benzylene.

In a preferred embodiment, Z₁ is:

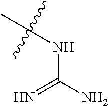

In a preferred embodiment, a compound of the invention is the compound named herein as 58.

The activity of the compounds of the invention means that they may be useful in the treatment of diseases in which NP-1 may have a significant role in pathology. The compounds of the invention may be useful for stimulating nerve repair, for the treatment of neurodegeneration and for use in anti-cancer therapy, for example in lung cancer. They may also be useful in the treatment of a disease where modulation of the immune system is required, for example, following transplant surgery. Yet other conditions that may be treated using a compound of the invention include skin diseases such as psoriasis, diseases requiring immunomodulation, angiogenesis in the eye, diabetes, macular degeneration, glaucoma, heart failure and Alzheimer's disease. Compounds of the invention may also be useful for the inhibition of platelet aggregation, and for the treatment of leukaemias and lymphomas and other diseases caused by HTLV1 infection.

Compounds of the invention may have utility in veterinary applications, in the therapy of liver disease, multiple sclerosis and in NRP-1-expressing tumours.

The compounds of the invention may be combined with another anti-cancer agent, such as avastin. They compounds of the invention may also be combined with an anti-angiogenic agents. The combination may be for separate, sequential or simultaneous use in therapy. The therapies are defined above.

For therapeutic use, compounds of the invention may be formulated and administered by procedures, and using components, known to those of ordinary skill in the art. The appropriate dosage of the compound may be chosen by the skilled person having regard to the usual factors such as the condition of the subject to be treated, the potency of the compound, the route of administration etc. Suitable routes of administration include oral, intravenous, intramuscular, intraperitoneal, intranasal and subcutaneous.

Without wishing to be bound by theory, a NP-1 antagonist may compete with semaphorin-3A for binding to NP-1, and thereby antagonise inhibitory effects of semaphorin-3A on axonal outgrowth and migration in nerve cells. Potential applications of this are in promoting neurite outgrowth, in stimulating nerve repair or treating neurodegeneration. Further, an NP-1 antagonist may promote the survival of semaphorin-3A-responsive neurones, an effect that would confirm or enhance its utility in the applications given above, and may extend these applications, e.g. to treating neuronal death caused by episodes of ischaemia as in stroke and some eye diseases.

Recent evidence suggests a role for NP-1 in angiogenesis. The evidence shows that NP-1 may be essential for VEGF-induced angiogenesis in cancer, eye disease, rheumatoid arthritis and other diseases. Therefore, NP-1 antagonists may have applications in the inhibition of VEGF-dependent angiogenesis in disease.

NP-1 antagonists may also play a role in modulating the immune system. Therefore, it may be useful to give a compound of the invention before, during or after a transplant.

In addition, a NP-1 antagonist may compete with VEGF for binding to NP-1 in tumour cells and promote cell death in NP-1-expressing tumour cells. Potential applications of this are in anti-cancer therapy. Furthermore, a NP-1 antagonist has anti-metastatic potential since it effectively inhibits carcinoma cell adhesion to extra-cellular matrix proteins and cell migration.

In a preferred embodiment, a compound of the invention may be used, together with a radionucleus or a paramagnetic nuclei (e.g. Gadolinium, with the appropriate type of chelate to complex the metal, well known to those skilled in the art), in radioimaging or as a contrast reagent in Magnetic Resonance Imaging.

The following examples illustrate the invention. General methods for the preparation of the compounds of the invention are given. Exemplified compounds are listed and are characterised by LC-MS. NP-1 binding data is also provided for some of the compounds.

### Definitions and Final Compound Characterisation

### Abbreviations

Arg, Arginine; eq, equivalents; Boc, tert-butoxy carbonyl; tBu, tert-butyl; DIPEA, N,N-diisopropylethylamine, HPLC, high performance liquid chromatography; LC-MS, liquid chromatography mass spectrometry Pbf, 2,2,4,6,7-pentamethyldihydrobenzofuran-5-sulfonyl; PG, protecting group; py, pyridine; PyBrOP, bromo-tris-pyrrolidino-phosphonium hexafluorophosphate; SCX-2, ISOLUTE SCX-2 strong cation exchange sorbent; TLC, thin-layer chromatography.

Preparative LC-MS: Mass-directed purification preparative LC-MS using a preparative C-18 column (Phenomenex Luna C18 (2), 100×21.2 mm, 5 μm).

Intermediate compounds, i.e. not assigned with a nominated number, were analysed by reverse-phase LC-MS (analytical C-18 column, Phenomenex Luna C18 (2), 50×3.0 mm, 3 μm) and an AB gradient of 5-95% for B, over 6.5 minutes, at a flow rate of 1.1 mL/minute, where eluent A was 0.1% formic acid/water and eluent B was 0.1% formic acid/acetonitrile or methanol.

All final compounds, i.e. assigned with a nominated number, were analysed by reverse-phase LC-MS (analytical C-18 column, Phenomenex Luna C18 (2), 150×4.6 mm, 5 μm) and an AB gradient of 5-95% for B, over 13 minutes, at a flow rate of 1.5 mL/minute, where eluent A was 0.1% formic acid/water and eluent B was 0.1% formic acid/acetonitrile or methanol

### 3-(5-Bromo-2,3-dihydro-benzofuran-7-sulfonylamino)-thiophene-2-carboxylic acid methyl ester

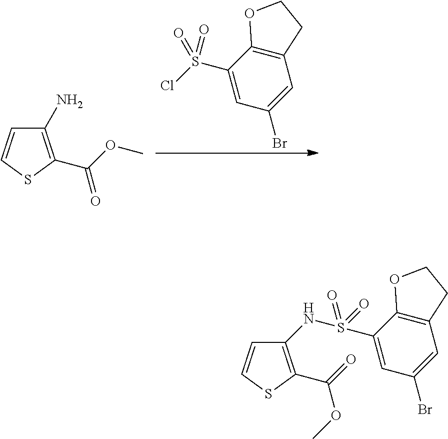

To a stirred solution of 5-Bromo-2,3-dihydro-benzofuran-7-sulfonyl chloride (1.25 eq, 2 g, 6.72 mmol) in pyridine (anhydrous, 10 mL), under nitrogen (balloon), at 20° C., was added methyl-3-aminothiophene-2-carboxylate (1 eq, 845 mg, 5.38 mmol) in pyridine (anhydrous, 5 mL), dropwise, over 120 minutes. The reaction mixture was stirred at 20° C. for 18 hours and after this time the reaction mixture was cooled (approx 0° C.) and water (5 mL) added dropwise. Precipitation occurred and the mixture was further diluted with water (20 mL) and the desired product collected by filtration, washed with ice-cold water (2×10 mL) and dried in vacuo to provide an off-white solid (2.2 g, 98%) which was used without further purification

LC-MS Rₜ 4.42 min.; purity 98%; MS m/z-416/418 [M−1]⁻.

### 3-(5-Bromo-2,3-dihydro-benzofuran-7-sulfonylamino)-thiophene-2-carboxylic acid

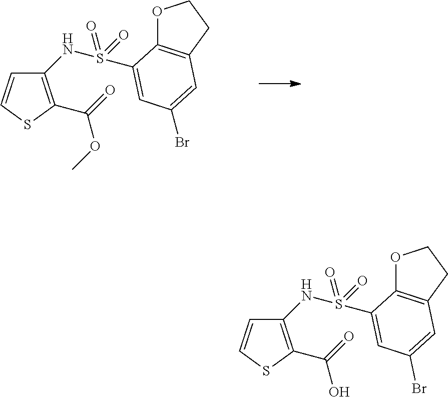

3-(5-Bromo-2,3-dihydro-benzofuran-7-sulfonylamino)-thiophene-2-carboxylic acid methyl ester (1 eq, 2.17 g, 5.2 mmol) was dissolved in tetrahydrofuran (20 mL) and methanol (12 mL). 1M Lithium hydroxide (5 eq, 26 mL, 26 mmol) was added as a single portion. The mixture was at stirred at 45° C. for 20 hours and after this time the organic solvents were removed in vacuo, the (aqueous) residue diluted with water (30 mL) and then acidified to pH 1 with 6M hydrochloric acid upon which precipitation occurred. The off-white solid was collected by suction filtration, washed with water (2×20 mL) and dried in vacuo to provide an off-white solid (1.8 g, 86%).

LC-MS Rₜ 4.54 min.; purity 95%; MS m/z-402/404 [M−1]⁻.

### (S)-2-{[3-(5-Bromo-2,3-dihydro-benzofuran-7-sulfonylamino)-thiophene-2-carbonyl]-amino}-5-(2,2,4,6,7-pentamethyl-2,3-dihydro-benzofuran-5-sulfonyl-guanidino)-pentanoic acid methyl ester

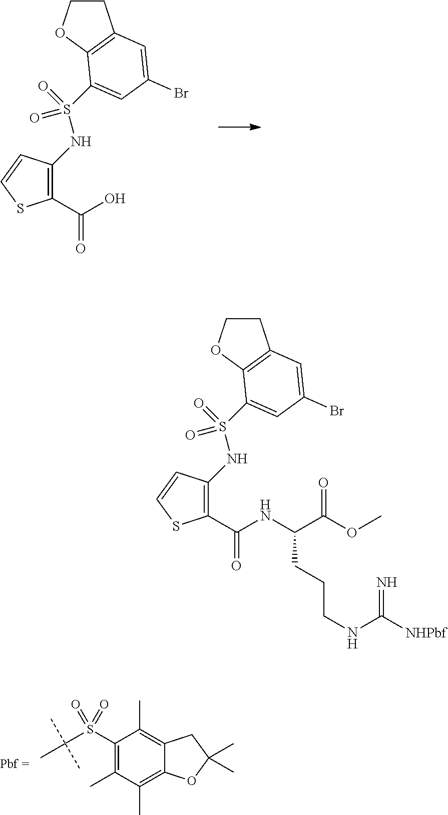

3-(5-Bromo-2,3-dihydro-benzofuran-7-sulfonylamino)-thiophene-2-carboxylic acid (1 eq, 2.11 g, 5.2 mmol) and bromo-tris-pyrrolidino-phosphonium hexafluorophosphate (PyBrOP; 1.1 eq, 2.66 g, 5.7 mmol) were suspended in dichloromethane (45 mL) and the mixture was stirred at 20° C. for 10 minutes. N,N-Diisopropylethylamine (7 eq, 6.34 mL, 36.4 mmol) was added to the mixture and stirred for a further 15 minutes. H-L-Arginine(Pbf)-OMe (hydrochloric acid salt; 1.1 eq, 7 g, 14.8 mmol) was added as a single portion and the reaction mixture (containing some white precipitate) was then stirred for 18 hours at 20° C. After this time the solvents were removed in vacuo and the resulting residue dissolved in ethyl acetate (60 mL) and partitioned with 1M hydrochloric acid (40 mL). The aqueous layer was separated and the organic layer was washed with further aliquots of 1M hydrochloric acid (3×40 mL). The organic layer was washed with brine (saturated, aqueous solution; 50 mL), dried over magnesium sulfate, filtered and the solvent removed in vacuo. The crude product (off-white foam; approx 4.5 g) was purified by flash column chromatography on silica gel (eluent: ethyl acetate/iso-hexane; 50:50, increasing to ethyl acetate only) affording the desired product as an off-white solid (3.38 g, 79%).

LC-MS Rₜ 4.77 min.; purity 95%; MS m/z-826/828 [M+1]⁺.

### (S)-2-{[3-(5-Bromo-2,3-dihydro-benzofuran-7-sulfonylamino)-thiophene-2-carbonyl]-amino}-5-(2,2,4,6,7-pentamethyl-2,3-dihydro-benzofuran-5-sulfonyl-guanidino)-pentanoic acid

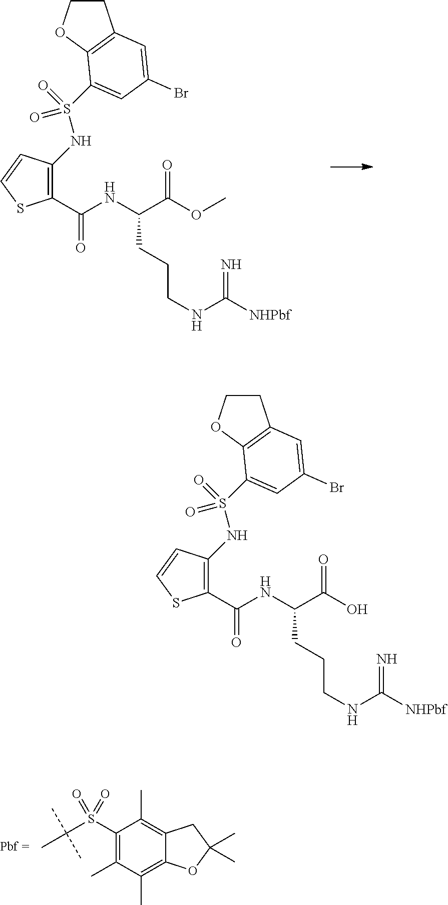

(S)-2-{[3-(5-Bromo-2,3-dihydro-benzofuran-7-sulfonylamino)-thiophene-2-carbonyl]-amino}-5-(2,2,4,6,7-pentamethyl-2,3-dihydro-benzofuran-5-sulfonyl-guanidino)-pentanoic acid methyl ester (1 eq, 2.45 g, 2.96 mmol) was stirred with 1M lithium hydroxide (5 eq, 14.82 mL mg, 14.82 mmol) in tetrahydrofuran (29 mL) at 20° C. for 3 hours. After this time the organic solvents were removed in vacuo, the (aqueous) residue diluted with water (30 mL) and then acidified to pH 1 with 6M hydrochloric acid. Ethyl acetate (200 mL) was added to the resulting suspension and, after thorough mixing, the organic layer separated. The aqueous layer was further extracted with ethyl acetate (150 mL) and the organic extracts were combined, washed with brine (saturated, aqueous solution; 3×75 mL), dried over magnesium sulfate, filtered and the solvent removed in vacuo. The product (pale yellow foam, 2.42 g, 100%) was used without further purification.

LC-MS Rₜ 4.89 min.; purity 90%; MS m/z-812/814 [M+1]⁺.

### General Procedure for Preparation of Elaborated Boronic Acids

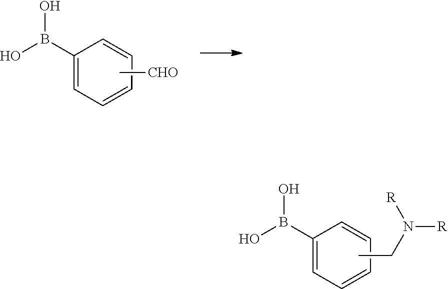

The appropriate formyl-phenylboronic acid (1.2 eq) and amine (1 eq) were combined and dissolved in dichloromethane (15 mL). Acetic acid (0.2 mL) was added and the reactions stirred at ambient temperature for 2 hours. At this time, sodium cyanoborohydride (2 eq) was added in a single portion and the reactions stirred for a further 20 hours at 20° C. The solvent was removed in vacuo and the crude residue dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/acetonitrile. The purified boronic acids were isolated via solvent evaporation and used without further purification.

TABLE X

| | |
|---|---|
| ID | 1-(2-Boronicacid-benzyl)-piperidine-4-carboxylic acid |
| | methyl ester. |
| Structure | 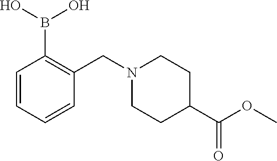 |
| Yield | White solid, 182 mg, 51% |
| LC-MS | Rₜ 1.50 min.; purity >95%; MS m/z − 278 [M + 1]⁺. |
| ID | [1-(2-Boronicacid-benzyl)-piperidine-4-yl]-acetic acid methyl |
| | ester. |
| Structure | 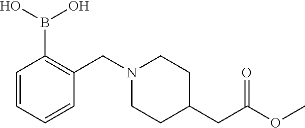 |
| Yield | White solid, 142 mg, 35% |
| LC-MS | Rₜ 1.70 min.; purity >95%; MS m/z − 292 [M + 1]⁺. |
| ID | 1-(3-Boronicacid-benzyl)-piperidine-4-carboxylic acid |
| | methyl ester. |
| Structure | 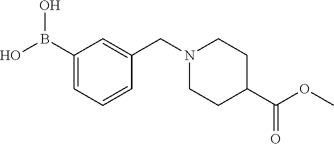 |
| Yield | White solid, 97 mg, 25% |
| LC-MS | Rₜ 0.35 min.; purity >90%; MS m/z − 278 [M + 1]⁺. |
| ID | [1-(3-Boronicacid-benzyl)- |
| | piperidine-4-yl]-acetic acid methyl ester. |
| Structure | 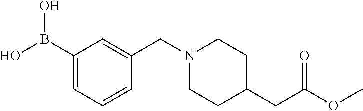 |
| Yield | White solid, 182 mg, 45% |
| LC-MS | Rₜ 1.25 min.; purity >90%; MS m/z − 292 [M + 1]⁺. |

### General Procedure for Solution-Phase Suzuki Coupling (Carboxylic Acids)

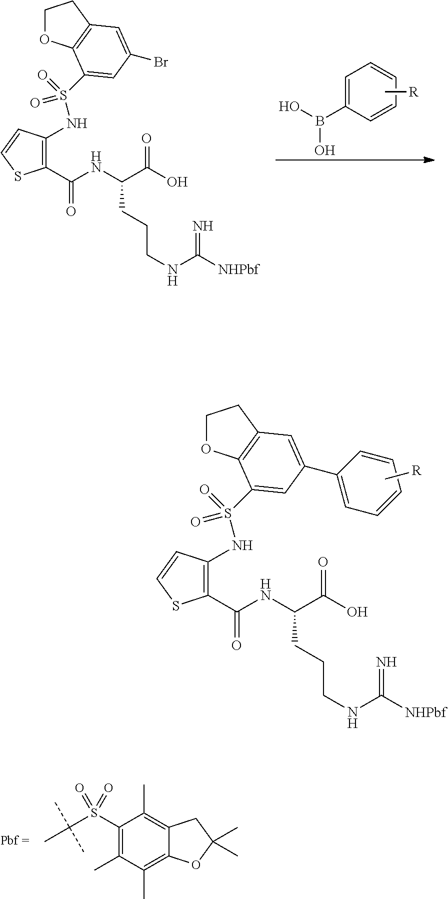

(S)-2-{[3-(5-Bromo-2,3-dihydro-benzofuran-7-sulfonylamino)-thiophene-2-carbonyl]-amino}-5-(2,2,4,6,7-pentamethyl-2,3-dihydro-benzofuran-5-sulfonyl-guanidino)-pentanoic acid (approx 1 g, 1.0 eq), corresponding boronic acid (1.5 eq) and tetrakis(triphenylphosphine)palladium(0) (0.05 eq) were suspended in degassed 1,2-dimethoxyethane (3 mL). Potassium phosphate (tribasic, 2 M aqueous solution, 4 eq), also degassed, was further added and the reaction mixture heated using microwave conditions (100 Watts, 90° C., ramp time=10 minutes). After this time the solvent was removed in vacuo and the resulting residue was partitioned between ethyl acetate (200 mL) and hydrochloric acid (1M aqueous solution; 150 mL). The phases were separated and the aqueous phase further extracted with ethyl acetate (200 mL). The organic extracts were combined, washed with brine (saturated, aqueous solution; 2×100 mL), dried over magnesium sulfate, filtered and the solvent removed in vacuo. The crude product (typically a yellow solid; approx 1.5 g) was purified by flash column chromatography on silica gel (eluent: dichloromethane increasing to dichloromethane/methanol; 75:25) to afford the desired products as summarised in Table 1.

TABLE 1

| | |
|---|---|
| ID | (S)-2-({3-[5-3-Formyl-phenyl)-2,3-dihydro-benzofuran-7- |
| | sulfonylamino]-thiophene-2-carbonyl}-amino)-5-guanidino(Pbf)- |
| | pentanoic acid. |
| Structure | 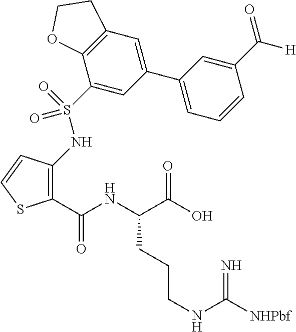 |
| Yield | Pale yellow solid, 779 mg, 84% |
| LC-MS | LC-MS Rₜ 4.76 min.; purity 89%; MS m/z − 838 [M + 1]⁺. |
| ID | (S)-2-({3-[5-2-Formyl-phenyl)-2,3-dihydro-benzofuran-7-sulfonylamino]- |
| | thiophene-2-carbonyl]-amino)-5-guanidino(Pbf)-pentanoic acid. |
| Structure | 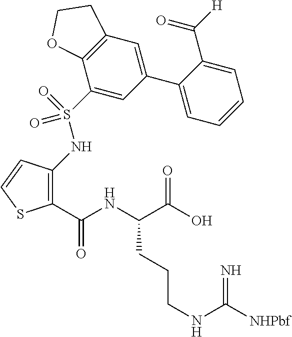 |
| Yield | Pale yellow solid, 810 mg, 77% |
| LC-MS | LC-MS Rₜ , 4.81 min.; purity 81%; MS m/z − 838 [M + 1]⁺. |
| ID | 1-(2-{7-[2-((S)-1-Carboxy-4-guanidino(Pbf)-butylcarbamoyl)- |
| | thiophen-3-ylsulfamoyl]-2,3-dihydro-benzofuran-5-yl}-benzyl)- |
| | piperidine-4-carboxylic acid methyl ester. |
| Structure | 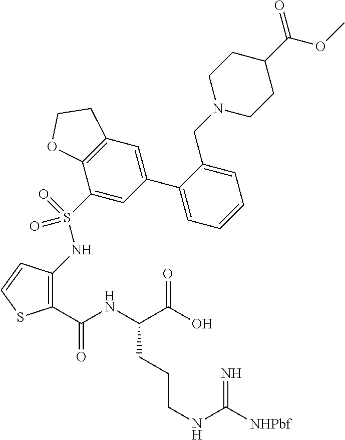 |
| Yield | Yellow solid, 87 mg, 82% |
| LC-MS | Rₜ 3.75 min.; purity >90%; MS m/z − 965 [M + 1]⁺. |
| ID | (S)-5-Guanidino(Pbf)-2-[(3-{5-[2-(4-methoxycarbonylmethyl-piperidin-1- |
| | ylmethyl)-phenyl]-2,3-dihydro-benzofuran-7-sulfonylamino}- |
| | thiophene-2-carbonyl)-amino]-pentanoic acid. |
| Structure | 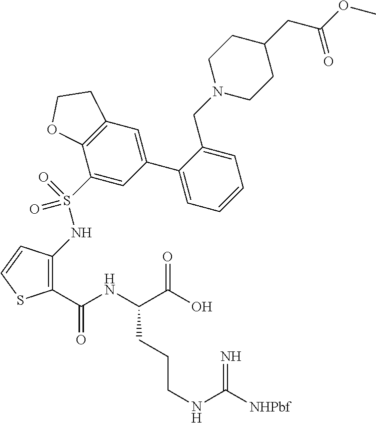 |
| Yield | Yellow solid, 86 mg, 80% |
| LC-MS | Rₜ 3.75 min.; purity > 90%; MS m/z − 979 [M + 1]⁺. |
| ID | 1-(3-{7-[2-((S)-1-Carboxy-4-guanidino(Pbf)-butylcarbamoyl)- |
| | thiophen-3-ylsulfamoyl]-2,3-dihydro-benzofuran-5-yl}-benzyl)- |
| | piperidine-4-carboxylic acid methyl ester. |
| Structure | 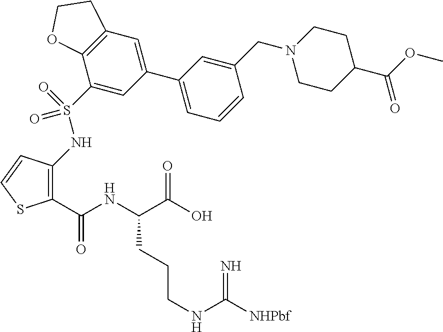 |
| Yield | Brown solid, 108 mg, 100% |
| LC-MS | Rₜ 3.65 min.; purity >90%; MS m/z − 965 [M + 1]⁺. |
| ID | (S)-5-Guanidino(Pbf)-2-[(3-{5-[3-(4-methoxycarbonylmethyl-piperidin-1- |
| | ylmethyl)-phenyl]-2,3-dihydro-benzofuran-7-sulfonylamino}- |
| | thiophene-2-carbonyl)-amino]-pentanoic acid. |
| Structure | 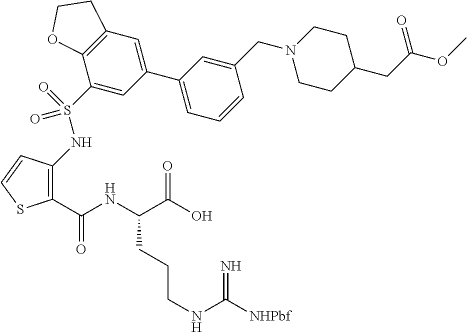 |
| Yield | Off white solid, 7 mg, 6% |
| LC-MS | Rₜ 3.69 min.; purity 77%; MS m/z − 979 [M + 1]⁺. |

### General Procedure for Methyl Ester Hydrolysis and Pbf Removal

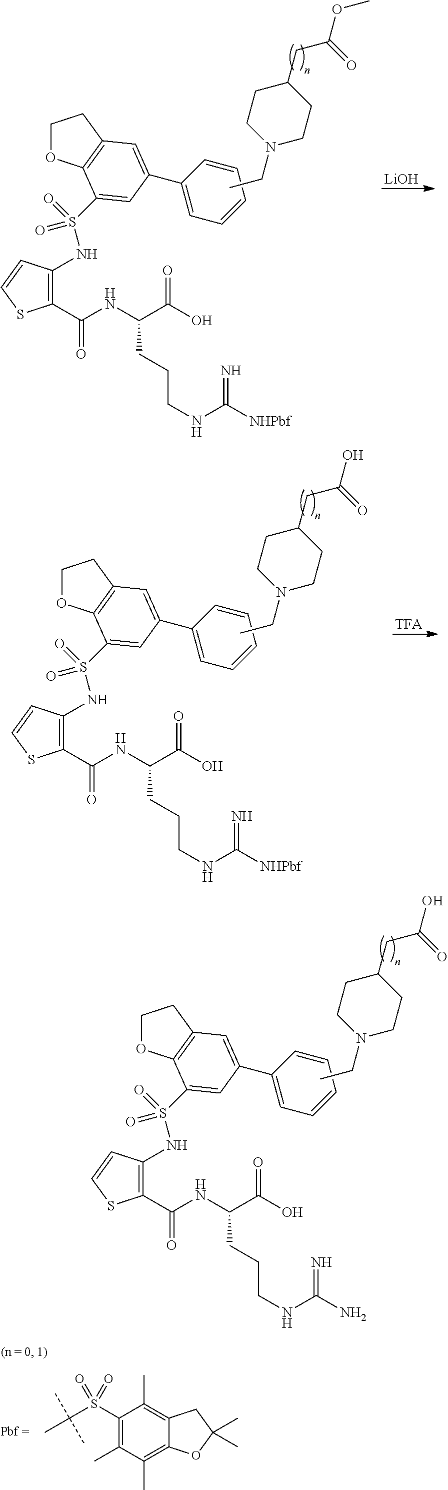

To a stirring suspension of methyl ester (1 eq) in 1,4-dioxane (1.2 mL) was added 1M lithium hydroxide (aqueous, 4 eq) and water (1.2 mL). The reaction was stirred at 20° C. for 24 hours whereupon the reaction was evaporated to dryness to give a white solid which was used without further purification.

The residue was dissolved in dichloromethane/trifluoroacetic acid (1:1, 5 mL) and stirred at room temperature for 1 hour. The solvent was removed in vacuo and the crude residue dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/acetonitrile. The purified peptidomimetics were isolated via solvent evaporation.

Table 2 summarises the final compounds constructed using these methods.

TABLE 2

| | |
|---|---|
| ID | 1; 1-(2-{7-[2-((S)-1-Carboxy-4-guanidino-butylcarbonyl)- |
| | thiophen-3-ylsulfamoyl]-2,3-dihydro-benzofuran-5-yl}-benzyl)- |
| | piperidine-4-carboxylic acid |
| Structure | 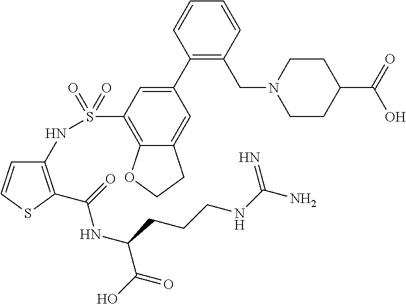 |
| Yield | White solid, 7.3 mg, 22% |
| LC-MS | Rₜ 4.62 min.; purity 100%; MS m/z − 699 [M + 1]^{+.} |
| ID | 2; (S)-2-[(3-{5-[2-(4-Carboxymethyl-piperidin-1-ylmethyl)-phenyl]-2,3- |
| | dihydro-benzofuran-7-sulfonylamino}-thiophene-2-carbonyl)-amino]-5- |
| | guanidino-pentanoic acid |
| Structure | 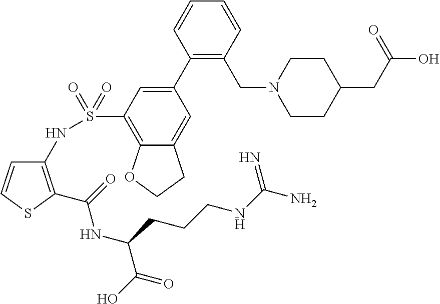 |
| Yield | Off white solid, 5.7 mg, 18% |
| LC-MS | Rₜ 4.62 min.; purity 90%; MS m/z − 713 [M + 1]⁺. |
| ID | 3; 1-(3-{7-[2-((S)-1-Carboxy-4-guanidino-butylcarbamoyl)- |
| | thiophen-3-ylsulfamoyl]-2,3-dihydro-benzofuran-5-yl}-benzyl)- |
| | piperidine-4-carboxylic acid |
| Structure | 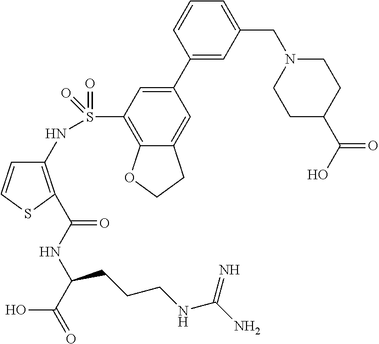 |
| Yield | White solid, 7.3 mg, 17% |
| LC-MS | Rₜ 4.5 min.; purity 100%; MS m/z − 699 [M + 1]⁺. |
| ID | 4; (S)-2-[(3-{5-[3-(4-Carboxymethyl-piperidin-1-ylmethyl)-phenyl]-2,3- |
| | dihydro-benzofuran-7-sulfonylamino}-thiophene-2-carbonyl)-amino]-5- |
| | guanidino-pentanoic acid |
| Structure | 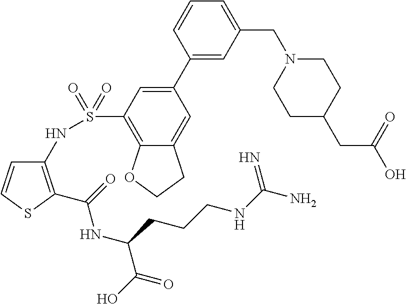 |
| Yield | White solid, 1.3 mg, 17% |
| LC-MS | Rₜ 5.16; purity 99%; MS m/z − 713 [M + 1]⁺. |

General Procedure for Pbf Removal

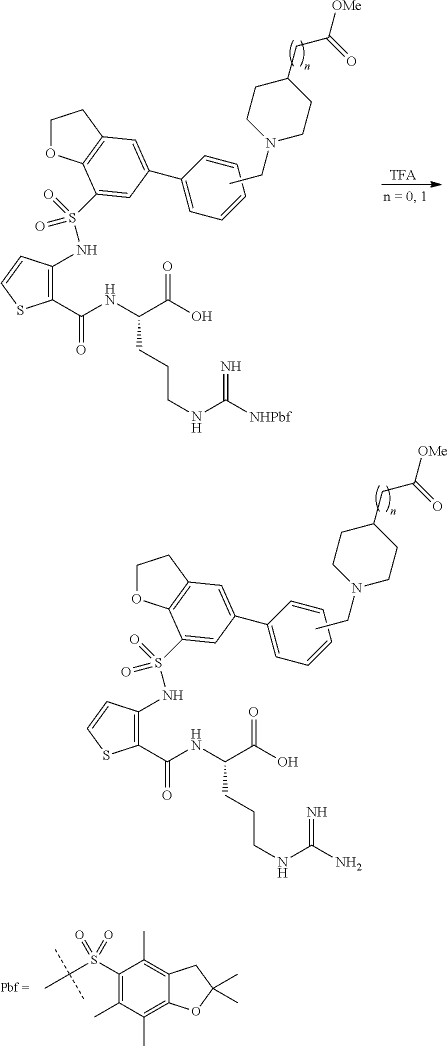

The residue was dissolved in dichloromethane/trifluoroacetic acid (1:1, 5 mL) and stirred at room temperature for 1 hour. The solvent was removed in vacuo and the crude residue dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/acetonitrile. The purified peptidomimetics were isolated via solvent evaporation.

Table 3 summarises the final compounds constructed using these methods.

TABLE 3

| | |
|---|---|
| ID | 5; 1-(2-{7-[2-((S)-1-Carboxy-4-guanidino-butylcarbamoyl)- |
| | thiophen-3-ylsulfamoyl]-2,3-dihydro-benzofuran-5-yl}-benzyl)- |
| | piperidine-4-carboxylic acid methyl ester |
| Structure | 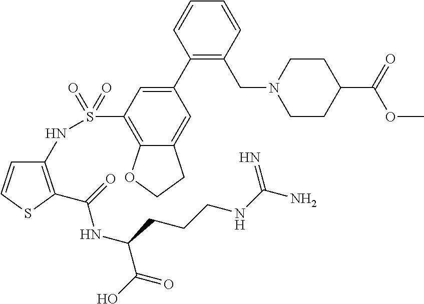 |
| Yield | White solid, 7.8 mg, 21% |
| LC-MS | Rₜ 4.6 min.; purity 99%; MS m/z − 713 [M + 1]⁺. |
| ID | 6; (S)-5-Guanidino-2-[(3-{5-[2-(4-methoxycarbonylmethyl-piperidin-1- |
| | ylmethyl)-phenyl]-2,3-dihydro-benzofuran- |
| | 7-sulfonylamino}-thiophene-2-carbonyl)-amino]-pentanoic acid |
| Structure | 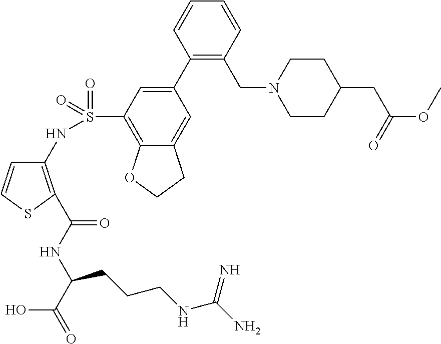 |
| Yield | White solid, 5.4 mg, 17% |
| LC-MS | Rₜ 4.76 min.: purity 88%; m/z − 727 [M + 1]⁺. |
| ID | 7; 1-(3-{7-[2-((S)-1-Carboxy-4-guanidino-butylcarbamoyl)- |
| | thiophen-3-ylsulfamoyl]-2,3-dihydro-benzofuran-5- |
| | yl}-benzyl)-piperidine-4-carboxylic acid methyl ester |
| Structure | 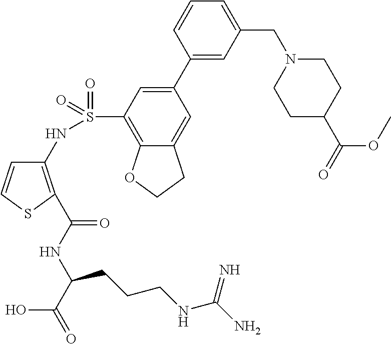 |
| Yield | White solid, 8 mg, 19% |
| LC-MS | Rₜ 4.59 min.; purity 86%; MS m/z − 713 [M + 1]⁺. |

### General Procedure for Reductive Amination and Pbf Removal

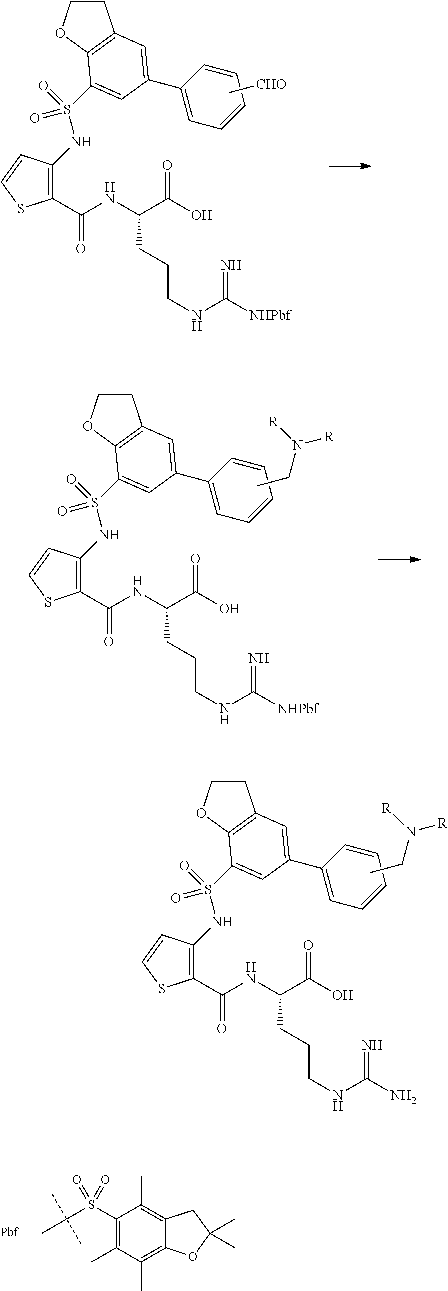

A solution of the aldehyde (1 eq) in tetrahydrofuran/methanol (1:1, 1.5 mL) was added to the amine (commercially available; 1.1 eq) followed by acetic acid (1-2 drops ˜pH6). The reaction was stirred at 20° C. for 2 hours before sodium cyanoborohydride (2 eq) in methanol (0.1 mL) was added in one portion. The reaction was stirred for a further 16 hours at 20° C. The reaction was filtered through a preconditioned SCX-2 (1 g) cartridge and the product eluted with 2M ammonia in methanol. Solvent evaporation gave the product as a yellow oil which was dissolved in dichloromethane/trifluoroacetic acid (1:1, 8 mL) and stirred at 20° C. for 1 hour. The solvent was removed in vacuo and the crude residue dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where, i. eluent A was 0.1% formic acid/water and eluent B was 0.1% formic acid/methanol or, ii, eluent A was 10 mM ammonium bicarbonate (pH9) and eluent B was 100% methanol. The purified peptidomimetics were isolated via solvent evaporation.

Table 4 summarises the final compounds constructed using these methods.

TABLE 4

| | |
|---|---|
| ID | 8; (S)-5-Guanidino-2-[(3-{5-[2-(4-pyridin-4-ylmethyl-piperazin-1- |
| | ylmethyl)-phenyl]-2,3-dihydro-benzofuran-7-sulfonylamino}- |
| | thiophene-2-carbonyl)-amino]-pentanoic acid |
| Structure | 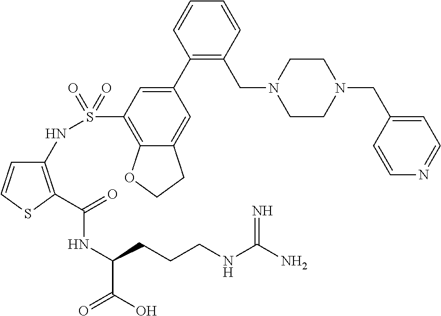 |
| Yield | White solid, 17.5 mg, 52% |
| LC-MS | Rₜ 4.54 min.; purity 100%; MS m/z − 747 [M + 1]⁺. |
| ID | 9; (S)-5-Guanidino-2-({3-[5-(2-{[(pyridin-3-ylmethyl)-amino]-methyl}- |
| | phenyl)-2,3-dihydro-benzofuran-7-sulfonylamino]-thiophene-2-carbonyl}- |
| | amino)-pentanoic acid |
| Structure | 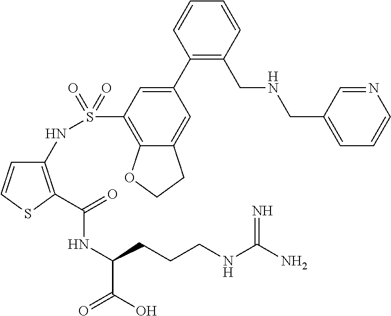 |
| Yield | White solid, 14 mg, 60% |
| LC-MS | Rₜ 4.42 min.; purity 100%; MS m/z − 678 [M + 1]⁺. |
| ID | 10; (S)-5-Guanidino-2-[(3-{5-[2-(4-pyridin-3-yl-piperazin-1-ylmethyl)- |
| | phenyl]-2,3-dihydro-benzofuran-7-sulfonylamino}-thiophene-2- |
| | carbonyl)-amino]-pentanoic acid |
| Structure | 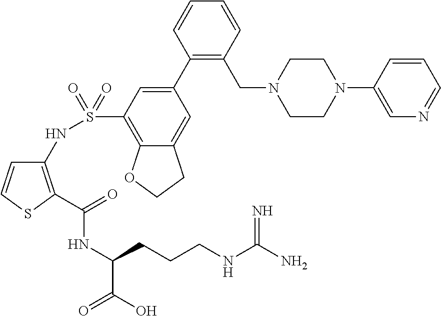 |
| | White solid, 4.7 mg, 33% |
| | Rₜ 4.54 min.; purity 100%; MS m/z − 747 [M + 1]⁺. |
| ID | 11; (S)-5-Guanidino-2-[(3-{5-[3-(4-pyridin-3-ylmethyl-piperazin-1- |
| | ylmethyl)-phenyl]-2,3-dihydro-benzofuran-7-sulfonylamino}- |
| | thiophene-2-carbonyl)-amino]-pentanoic acid |
| Structure | 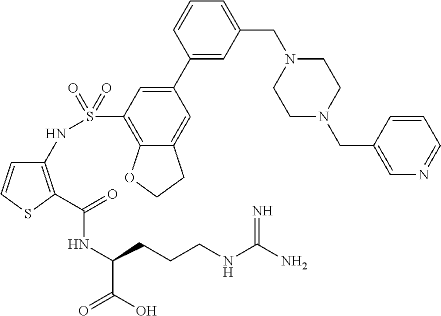 |
| | White solid, 10.2 mg, 25% |
| | Rₜ 4.14 min.; purity 100%; MS m/z − 747 [M + 1]⁺. |
| ID | 12; (S)-5-Guanidino-2-({3-[5-(3-{[(pyridin-3-ylmethyl)-amino]- |
| | methyl}-phenyl)-2,3-dihydro-benzofuran-7-sulfonyl- |
| | amino]-thiophene-2-carbonyl}-amino)-pentanoic acid |
| Structure | 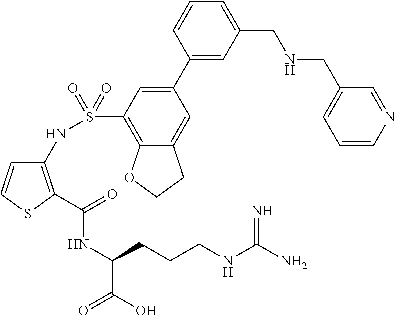 |
| Yield | White solid, 5.7 mg, 26% |
| LC-MS | Rₜ 4.43 min.; purity 100%; MS m/z − 678 [M + 1]⁺. |
| ID | 13; (S)-5-Guanidino-2-[(3-{5-[3-(4-pyridin-3-yl-piperazin-1-ylmethyl)- |
| | phenyl]-2,3-dihydro-benzofuran-7-sulfonylamino}-thiophene-2-carbonyl)- |
| | amino]-pentanoic acid |
| Structure | 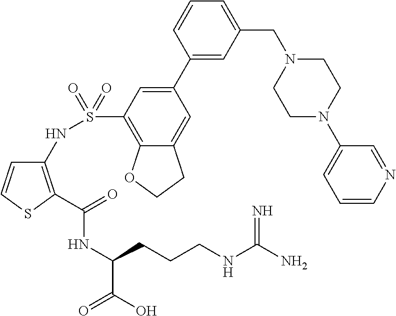 |
| Yield | White solid, 7.3 mg, 35% |
| LC-MS | Rₜ 3.95 min.; purity 100%; MS m/z − 733 [M + 1]⁺. |
| ID | 14; (S)-5-Guanidino-2-[(3-{5-[2-(4-pyridin-2-yl-piperazin-1-ylmethyl)- |
| | phenyl]-2,3-dihydro-benzofuran-7-sulfonylamino}-thiophene-2- |
| | carbonyl)-amino]-pentanoic acid |
| Structure | 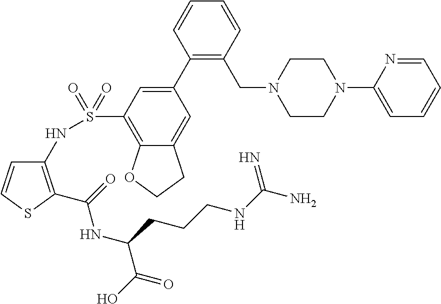 |
| Yield | White solid, 14.3 mg, 44% |
| LC-MS | Rₜ 4.94 min.; purity 96; MS m/z − 733 [M + 1]⁺. |
| ID | 15; (S)-5-Guanidino-2-[(3-{5-[2-(4-pyridin-4-yl-piperazin-1-ylmethyl)- |
| | phenyl]-2,3-dihydro-benzofuran-7-sulfonylamino}-thiophene-2-carbonyl)- |
| | amino]-pentanoic acid |
| Structure | 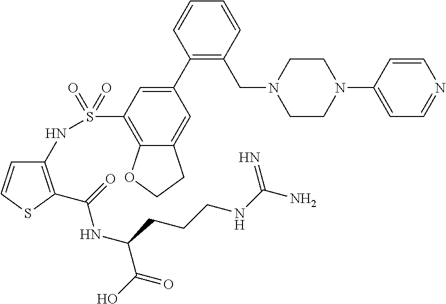 |
| Yield | White solid, 16.4 mg, 50% |
| LC-MS | Rₜ 4.42 min.; purity 99%; MS m/z − 733 [M + 1]⁺. |
| ID | 16; (S)-5-Guanidino-2-[(3-{5-[2-(4-pyridin-2-ylmethyl-piperazin-1- |
| | ylmethyl)-phenyl]-2,3-dihydro-benzofuran-7- |
| | sulfonylamino}-thiophene-2-carbonyl)-amino]-pentanoic acid |
| Structure | 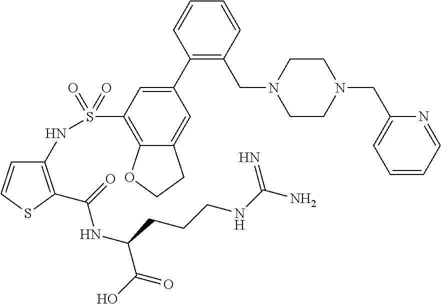 |
| Yield | White solid, 10.4 mg, 31% |
| LC-MS | Rₜ 4.66 min.; purity 99%; MS m/z − 747 [M + 1]⁺. |
| ID | 17; (S)-5-Guanidino-2-[(3-{5-[2-(4-pyridin-4-ylmethyl-piperazin-1- |
| | ylmethyl)-phenyl]-2,3-dihydro-benzofuran-7-sulfonylamino}- |
| | thiophene-2-carbonyl)-amino]-pentanoic acid |
| Structure | 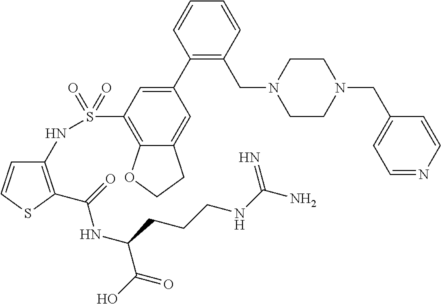 |
| Yield | White solid, 8.3 mg, 25% |
| LC-MS | Rₜ 4.11 min.; purity 99%; MS m/z − 747 [M + 1]⁺. |
| ID | 18; (S)-5-Guanidino-2-({3-[5-(2-{[(pyridin-2-ylmethyl)-amino]- |
| | methyl}-phenyl)-2,3-dihydro-benzofuran-7-sulfon |
| | ylamino]-thiophene-2-carbonyl}-amino)-pentanoic acid |
| Structure | 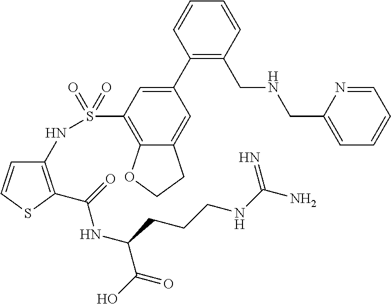 |
| Yield | White solid, 6.3 mg, 21% |
| LC-MS | Rₜ 4.62 min.; purity 95%; MS m/z − 678 [M + 1]⁺. |
| ID | 19; (S)-5-Guanidino-2-({3-[5-(2-{[(pyridin-4-ylmethyl)-amino]-methyl}- |
| | phenyl)-2,3-dihydro-benzofuran-7-sulfon |
| | ylamino]-thiophene-2-carbonyl}-amino)-pentanoic acid |
| Structure | 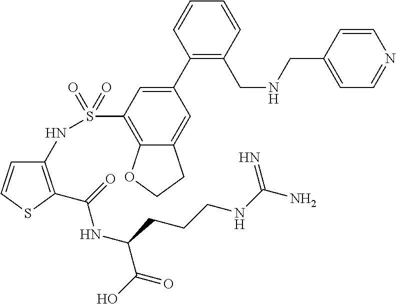 |
| Yield | White solid, 5.5 mg, 18% |
| LC-MS | Rₜ 4.33 min.; purity 98%; MS m/z − 678 [M + 1]⁺. |
| ID | 20; (S)-5-Guanidino-2-({3-[5-(2-piperazin-1-ylmethyl-phenyl)-2,3- |
| | dihydro-benzofuran-7-sulfonylamino]-thiophene-2- |
| | carbonyl}-amino)-pentanoic acid |
| Structure | 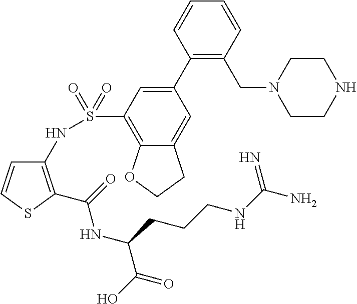 |
| Yield | White solid, 14.8 mg, 50% |
| LC-MS | Rₜ 4.95 min.; purity 92%; MS m/z − 656 [M + 1]⁺. |
| ID | 21; (S)-2-[(3-{5-[2-(4-Amino-piperidin-1-ylmethyl)-phenyl]-2,3-dihydro- |
| | benzofuran-7-sulfonylamino}-thiophene-2-carbonyl)-amino]-5- |
| | guanidino-pentanoic acid |
| Structure | 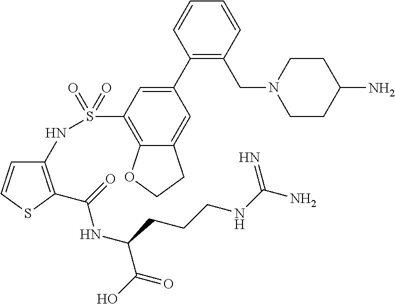 |
| Yield | White solid, 8.8 mg, 30% |
| LC-MS | Rₜ 3.71 min.; purity 91%; MS m/z − 670 [M + 1]⁺. |
| ID | 22; (S)-5-Guanidino-2-[(3-{5-[3-(4-morpholin-4-yl-piperidin-1- |
| | ylmethyl)-phenyl]-2,3-dihydro-benzofuran-7-sulfonylamino}- |
| | thiophene-2-carbonyl)-amino]-pentanoic acid |
| Structure | 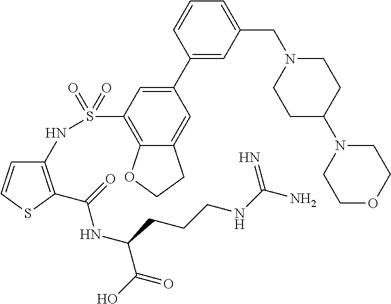 |
| Yield | White solid, 11.2 mg, 39% |
| LC-MS | Rₜ 4.42 min.; purity 98%; MS m/z − 631 [M + 1]⁺. |
| ID | 23; (S)-5-Guanidino-2-({3-[5-(2-{[(3-methyl-3H-imidazol-4-ylmethyl)- |
| | amino]-methyl}-phenyl)-2,3-dihydro-benzofuran-7-sulfonylamino]- |
| | thiophene-2-carbonyl}-amino)-pentanoic acid |
| Structure | 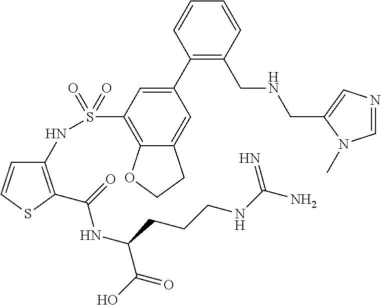 |
| Yield | White solid, 11.5 mg, 38% |
| LC-MS | Rₜ 3.88 min.; purity 98%; MS m/z − 681 [M + 1]⁺. |
| ID | 24; (S)-5-Guanidino-2-({3-[5-(2-{[(1H-imidazol-2-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 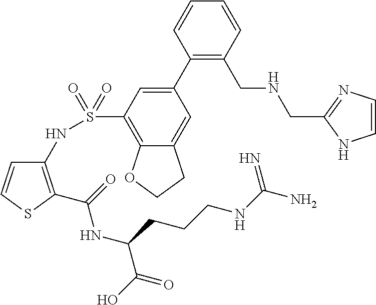 |
| Yield | White solid, 8.9 mg, 30% |
| LC-MS | Rₜ 4.48 min.; purity 98%; MS m/z − 667 [M + 1]⁺. |
| ID | 25; (S)-5-Guanidino-2-[(3-{5-[2-(4-morpholin-4-yl-piperidin-1-ylmethyl)- |
| | phenyl]-2,3-dihydro-benzofuran-7-sulfonylamino}-thiophene-2-carbonyl)- |
| | amino]-pentanoic acid |
| Structure | 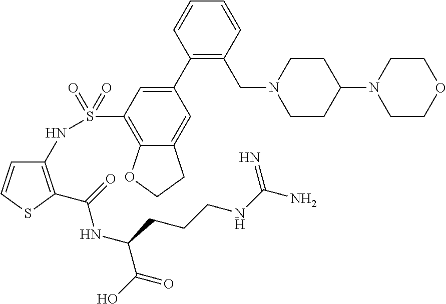 |
| Yield | White solid, 5.8 mg, 18% |
| LC-MS | Rₜ 3.84 min.; purity 96%; MS m/z − 640 [M + 1]⁺. |
| ID | 26; (S)-5-Guanidino-2-({3-[5-(2-{[(5-methyl-isoxazol-3-ylmethyl)-amino]- |
| | methyl}-phenyl)-2,3-dihydro-benzofuran-7-sulfonylamino]-thiophene-2- |
| | carbonyl}-amino)-pentanoic acid |
| Structure | 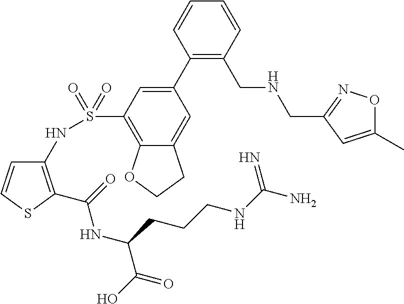 |
| Yield | White solid, 13.2 mg, 43% |
| LC-MS | Rₜ 4.68 min.; purity 95%; MS m/z − 682 [M + 1]⁺. |
| ID | 27; (S)-5-Guanidino-2-[(3-{5-[3-(4-pyridin-2-yl-piperazin-1-ylmethyl)- |
| | phenyl]-2,3-dihydro-benzofuran-7-sulfonylamino}-thiophene-2-carbonyl)- |
| | amino]-pentanoic acid |
| Structure | 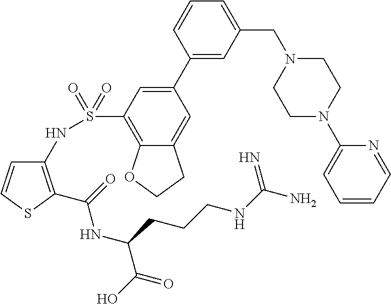 |
| Yield | White solid, 10.3 mg, 31% |
| LC-MS | Rₜ 4.66 min.; purity 100%; MS m/z − 733 [M + 1]⁺. |
| ID | 28; (S)-5-Guanidino-2-[(3-{5-[3-(4-pyridin-4-yl-piperazin-1-ylmethyl)- |
| | phenyl]-2,3-dihydro-benzofuran-7-sulfonylamino}-thiophene-2- |
| | carbonyl)-amino]-pentanoic acid |
| Structure | 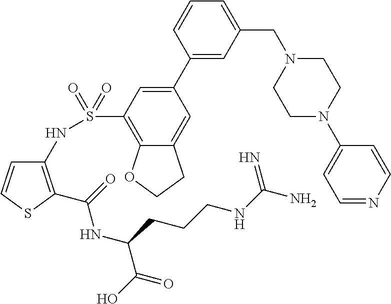 |
| Yield | White solid, 11.3 mg, 35% |
| LC-MS | Rₜ 3.94 min.; purity 100%; MS m/z − 733 [M + 1]⁺. |
| ID | 29; (S)-5-Guanidino-2-[(3-{5-[3-(4-pyridin-2-ylmethyl-piperazin-1- |
| | ylmethyl)-phenyl]-2,3-dihydro-benzofuran-7-sulfonylamino}- |
| | thiophene-2-carbonyl)-amino]-pentanoic acid |
| Structure | 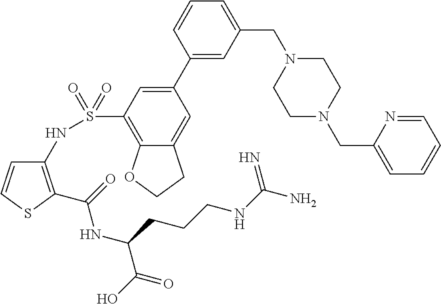 |
| Yield | White solid, 11.9 mg, 36% |
| LC-MS | Rₜ 4.36 min.; purity 100%; MS m/z − 747 [M + 1]⁺. |
| ID | 30; (S)-5-Guanidino-2-[(3-{5-[3-(4-pyridin-4-ylmethyl-piperazin-1- |
| | ylmethyl)-phenyl]-2,3-dihydro-benzofuran-7- |
| | sulfonylamino}-thiophene-2-carbonyl)-amino]-pentanoic acid |
| Structure | 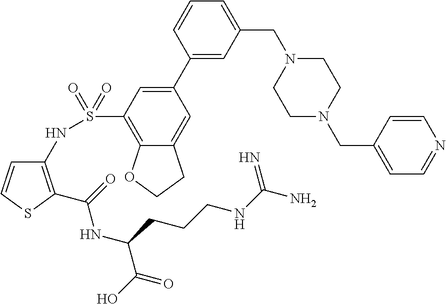 |
| Yield | White solid, 11.7 mg, 35% |
| LC-MS | Rₜ 3.94 min.; purity 100%; MS m/z − 747 [M + 1]⁺. |
| ID | 31; (S)-5-Guanidino-2-({3-[5-(3-{[(pyridin-2-ylmethyl)-amino]-methyl}- |
| | phenyl)-2,3-dihydro-benzofuran-7-sulfonylamino]-thiophene-2-carbonyl}- |
| | amino)-pentanoic acid |
| Structure | 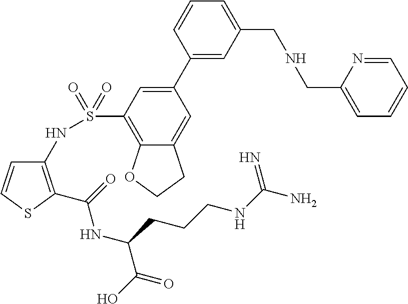 |
| Yield | White solid, 8.8 mg, 31% |
| LC-MS | Rₜ 4.72 min.; purity 85%; MS m/z − 678 [M + 1]⁺. |
| ID | 32; (S)-5-Guanidino-2-({3-[5-(3-{[(pyridin-4-ylmethyl)-amino]- |
| | methyl}-phenyl)-2,3-dihydro-benzofuran-7-sulfon |
| | ylamino]-thiophene-2-carbonyl}-amino)-pentanoic acid |
| Structure | 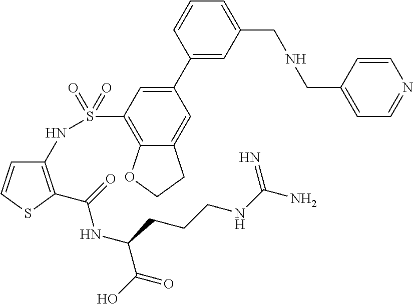 |
| Yield | White solid, 5.5 mg, 18% |
| LC-MS | Rₜ 4.29 min.; purity 100%; MS m/z − 678 [M + 1]⁺. |
| ID | 33; (S)-5-Guanidino-2-({3-[5-(3-piperazin-1-ylmethyl-phenyl)-2,3- |
| | dihydro-benzofuran-7-sulfonylamino]-thiophene-2-carbonyl}-amino)- |
| | pentanoic acid |
| Structure | 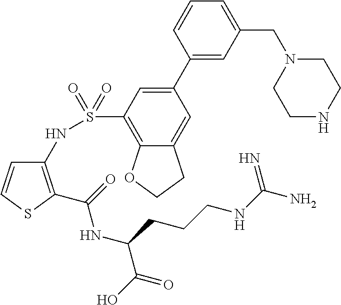 |
| Yield | White solid, 7.1 mg, 24% |
| LC-MS | Rₜ 4.44 min.; purity 100%; MS m/z − 656 [M + 1]⁺. |
| ID | 34; (S)-2-[(3-{5-[3-(4-Amino-piperidin-1-ylmethyl)-phenyl]-2,3- |
| | dihydro-benzofuran-7-sulfonylamino}-thiophene- |
| | 2-carbonyl)-amino]-5-guanidino-pentanoic acid |
| Structure | 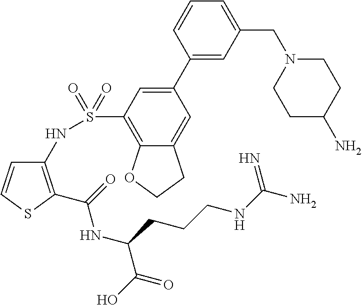 |
| Yield | White solid, 11.7 mg, 39% |
| LC-MS | Rₜ 3.64 min.; purity 99%; MS m/z − 670 [M + 1]⁺. |
| ID | 35; (S)-5-Guanidino-2-{[3-(5-{3-[(2-hydroxy-ethylamino)-methyl]-phenyl}- |
| | 2,3-dihydro-benzofuran-7-sulfonylamino)-thiophene-2-carbonyl]- |
| | amino}-pentanoic acid |
| Structure | 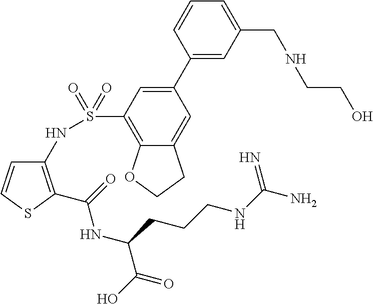 |
| Yield | White solid, 9.1 mg, 32% |
| LC-MS | Rₜ 4.30 min.; purity 100%; MS m/z − 631 [M + 1]⁺. |
| ID | 36; (S)-5-Guranidino-2-({3-[5-(3-{[(3-methyl-3H-imidazol-4-ylmethyl)- |
| | amino]-methyl}-phenyl)-2,3-dihydro-benzofuran-7- |
| | sulfonylamino]-thiophene-2-carbonyl}-amino)-pentanoic acid |
| Structure | 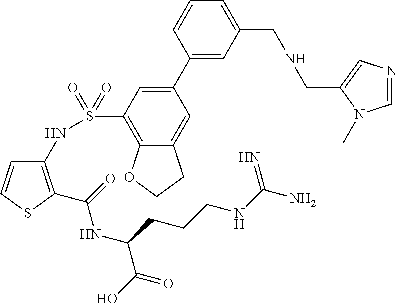 |
| Yield | White solid, 13.6 mg, 45% |
| LC-MS | Rₜ 3.7 min.; purity 100%; MS m/z − 681 [M + 1]⁺. |
| ID | 37; (S)-5-Guanidino-2-({3-[5-3-{[(1H-imidazol-2-ylmethyl)-amino]-methyl}- |
| | phenyl)-2,3-dihydro-benzofuran-7-sulfonylamino]-thiophene-2-carbonyl}- |
| | amino)-pentanoic acid |
| Structure | 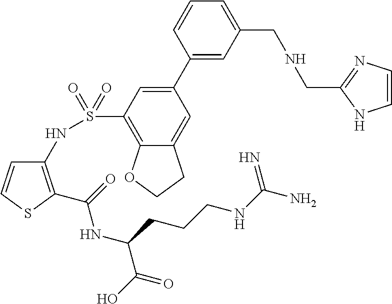 |
| Yield | White solid, 9.6 mg, 32% |
| LC-MS | Rₜ 4.30 min.; purity 97%; MS m/z − 667 [M + 1]⁺. |
| ID | 38; (S)-5-Guanidino-2-[(3-{5-[3-(4-morpholin-4-yl-piperidin-1- |
| | ylmethyl)-phenyl]-2,3-dihydro-benzofuran-7-sulfonylamino}- |
| | thiophene-2-carbonyl)-amino]-pentanoic acid |
| Structure | 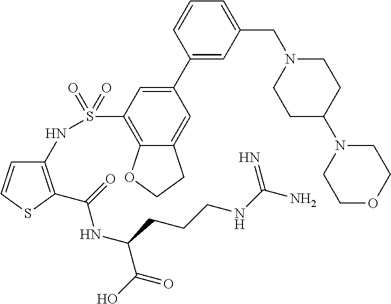 |
| Yield | White solid, 9.5 mg, 29% |
| LC-MS | Rₜ 3.72 min.; purity 100%; MS m/z − 740 [M + 1]⁺. |
| ID | 39; (S)-2-({3-[5-(2-{[(6-Amino-pyridin-3-ylmethyl)-amino]-methyl}-phenyl)- |
| | 2,3-dihydro-benzofuran-7-sulfonylamino]-thiophene-2-carbonyl}- |
| | amino)-5-guanidino-pentanoic acid |
| Structure | 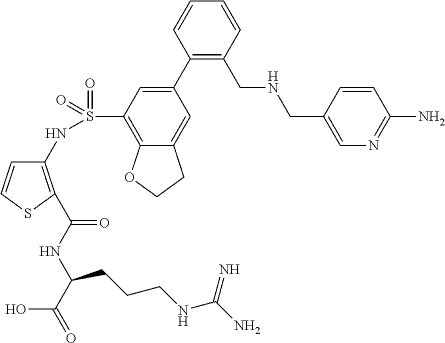 |
| Yield | White solid, 10.9 mg, 35% |
| LC-MS | Rₜ 3.75 min.; purity 100%; MS m/z − 693 [M + 1]⁺. |
| ID | 40; (S)-2-({3-[5-(3-{[(6-Amino-pyridin-3-ylmethyl)-amino]-methyl}- |
| | phenyl)-2,3-dihydro-benzofuran-7sulfonyl |
| | amino]-thiophene-2-carbonyl}-amino)-5-guanidino-pentanoic acid |
| Structure | 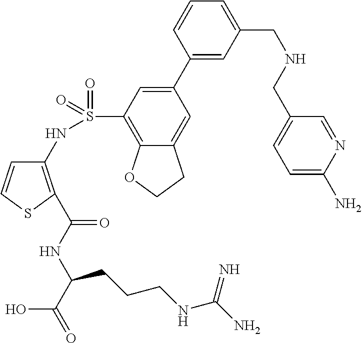 |
| Yield | White solid, 17.9 mg, 58% |
| LC-MS | Rₜ 3.62 min.; purity 89%; MS m/z − 693 [M + 1]⁺. |
| ID | 41; (S)-5-Guanidino-2-({3-[5-(3-{[3-(5-methyl-1H-pyrazol-4-yl)-propylamino]- |
| | methyl}-phenyl)-2,3-dihydro-benzofuran-7-sulfonylamino]- |
| | thiophene-2-carbonyl}-amino)-pentanoic acid |
| Structure | 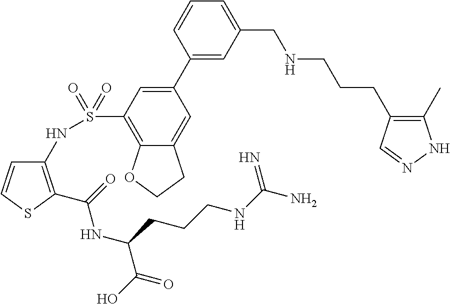 |
| Yield | White solid, 11.9 mg, 38% |
| LC-MS | Rₜ 4.46 min.; purity 100%; MS m/z − 709 [M + 1]⁺. |
| ID | 42; (S)-5-Guanidino-2-({3-[5-(3-{[2-(1H-pyrazol-4-yl)-ethylamino]- |
| | methyl}-phenyl)-2,3-dihydro-benzofuran-7-sulfonylamino]- |
| | thiophene-2-carbonyl}-amino)-pentanoic acid |
| Structure | 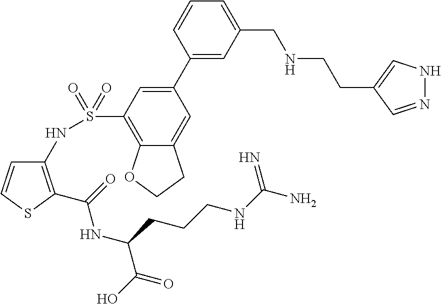 |
| Yield | White solid, 14 mg, 46% |
| LC-MS | Rₜ 4.33 min.; purity 93%; MS m/z − 681 [M + 1]⁺. |
| ID | 43; (S)-5-Guanidino-2-({3-[5-(3-{[(6-hydroxy-pyridin-3-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 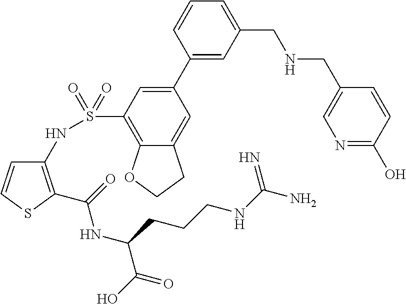 |
| Yield | White solid, 6 mg, 20% |
| LC-MS | Rₜ 4.21 min.; purity 83%; MS m/z − 694 [M + 1]⁺. |
| ID | 44; (S)-5-Guanidino-2-({3-[5-(3-{[3-(5-methyl-pyridin-2-ylamino)- |
| | propylamino]-methyl}-phenyl)-2,3-dihydro-benzofuran-7- |
| | sulfonylamino]-thiophene-2-carbonyl}-amino)-pentanoic acid |
| Structure | 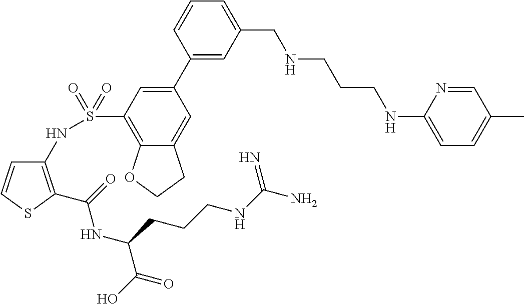 |
| Yield | White solid, 13.1 mg, 40% |
| LC-MS | Rₜ 3.90 min.; purity 100%; MS m/z − 735 [M + 1]⁺. |
| ID | 45; (S)-5-Guanidino-2-({3-[5-(3-{[2-(4-methyl-pyridin-2-ylamino)- |
| | ethylamino]-methyl}-phenyl)-2,3-dihydro-benzofuran-7-sulfonylamino]- |
| | thiophene-2-carbonyl}-amino)-pentanoic acid |
| Structure | 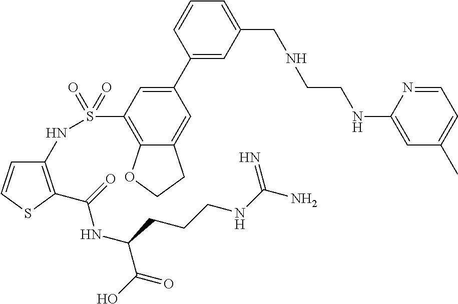 |
| Yield | White solid, 8.2 mg, 26% |
| LC-MS | Rₜ 4.02 min.; purity 100%; MS m/z − 721 [M + 1]⁺. |
| ID | 46; (S)-5-Guanidino-2-({3-[5-(2-{[3-(5-methyl-1H-pyrazol-4-yl)-propylamino]- |
| | methyl}-phenyl)-2,3-dihydro-benzofuran-7-sulfonylamino]- |
| | thiophene-2-carbonyl}-amino)-pentanoic acid |
| Structure | 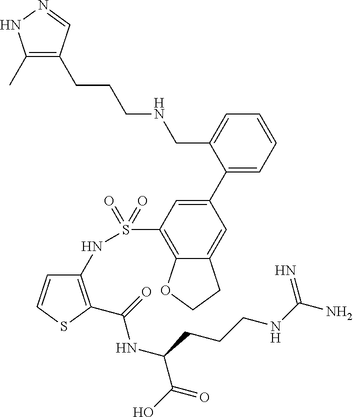 |
| Yield | White solid, 3.7 mg, 12% |
| LC-MS | Rₜ 4.49 min.; purity 96%; MS m/z − 709 [M + 1]⁺. |
| ID | 47; (S)-5-Guanidino-2-({3-[5-(2-{[2-(1H-pyrazol-4-yl)-ethylamino]- |
| | methyl}-phenyl)-2,3-dihydro-benzofuran-7-sulfonylamino]- |
| | thiophene-2-carbonyl}-amino)-pentanoic acid |
| Structure | 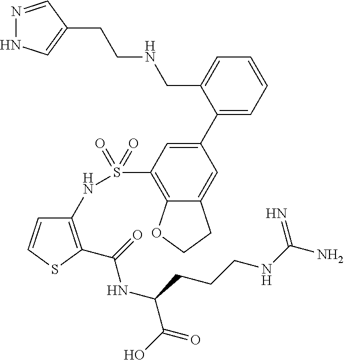 |
| Yield | White solid, 5 mg, 17% |
| LC-MS | Rₜ 4.40 min.; purity 87%; MS m/z − 681 [M + 1]⁺. |
| ID | 48: (S)-5-Guanidino-2-({3-[5-(3-{[(5-methyl-isoxazol-3-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 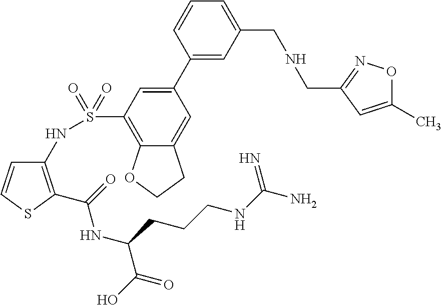 |
| Yield | White solid, 7.8 mg, 25% |
| LC-MS | Rₜ 4.78 min.; purity 94%; MS m/z − 682 [M + 1]⁺. |
| ID | 49; (S)-5-Guanidino-2-({3-[5-(2-{[(6-hydroxy-pyridin-3-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 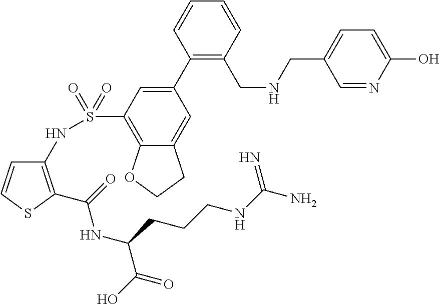 |
| Yield | White solid, 2 mg, 6% |
| LC-MS | Rₜ 4.28 min.; purity 93%; MS m/z − 694 [M + 1]⁺. |
| ID | 50; (S)-5-Guanidino-2-({3-[5-(2-{[3-(5-methyl-pyridin-2-ylamino)- |
| | propylamino]-methyl}-phenyl)-2,3-dihydro-benzofuran-7-sulfonylamino]- |
| | thiophene-2-carbonyl}-amino)-pentanoic acid |
| Structure | 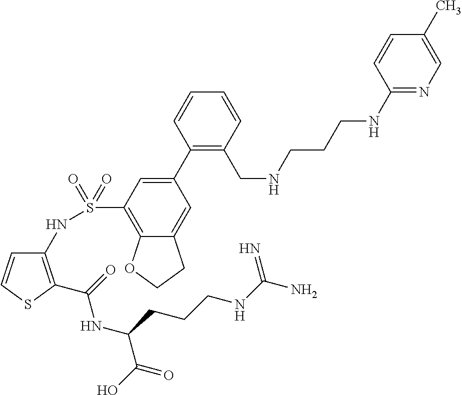 |
| Yield | White solid, 5.8 mg, 18% |
| LC-MS | Rₜ 4.08 min.; purity 99%; MS m/z − 735 [M + 1]⁺. |
| ID | 51; (S)-5-Guanidino-2-({3-[5-(3-{[(1H-pyrazol-4-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 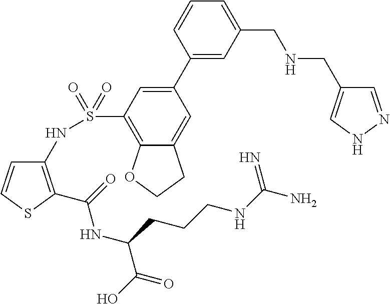 |
| Yield | Off white solid, 11.2 mg, 40% |
| LC-MS | Rₜ 4.38 min.; purity 94%; MS m/z − 667 [M + 1]⁺. |
| ID | 52; (S)-5-Guanidino-2-({3-[5-(3-{[(1H-pyrazol-3-ylmethyl)-amino]-methyl}- |
| | phenyl)-2,3-dihydro-benzofuran-7-sulfonylamino]-thiophene-2-carbonyl}- |
| | amino)-pentanoic acid |
| Structure | 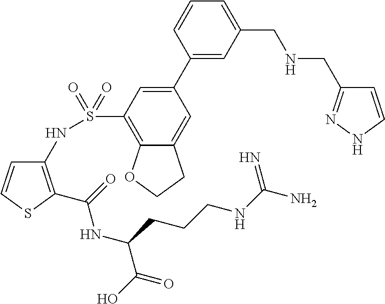 |
| Yield | White solid, 13.1 mg, 48% |
| LC-MS | Rₜ 4.46 min.; purity 92%; MS m/z − 667 [M + 1]⁺. |
| ID | 53; (S)-5-Guanidino-2-({3-[5-(2-{[(1H-pyrazol-3-ylmethyl)-amino]- |
| | methyl}-phenyl)-2,3-dihydro-benzofuran-7-sulfonylamino]- |
| | thiophene-2-carbonyl}-amino)-pentanoic acid |
| Structure | 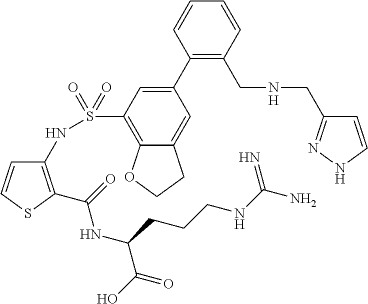 |
| Yield | White solid, 12.5 mg, 44% |
| LC-MS | Rₜ 4.44 min.; purity 94%; MS m/z − 667 [M + 1]⁺. |
| ID | 54; (S)-5-Guanidino-2-({3-[5-(2-{[(1H-pyrazol-4-ylmethyl)-amino]-methyl}- |
| | phenyl)-2,3-dihydro-benzofuran-7-sulfonylamino]-thiophene-2-carbonyl}- |
| | amino)-pentanoic acid |
| Structure | 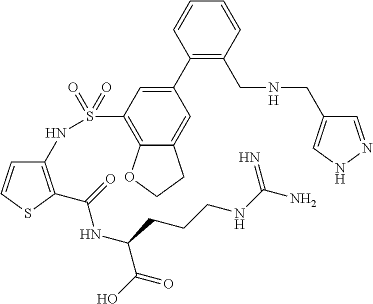 |
| Yield | White solid, 8.6 mg, 30% |
| LC-MS | Rₜ 4.40 min.; purity 96%; MS m/z − 667 [M + 1]⁺. |
| ID | 55; (S)-5-Guanidino-2-{[3-(5-{3-[4-(2-hydroxy-ethyl)-piperazin-1- |
| | ylmethyl]-phenyl}-2,3-dihydro-benzofuran-7-sulfonylamino)- |
| | thiophene-2-carbonyl]-amino}-pentanoic acid |
| Structure | 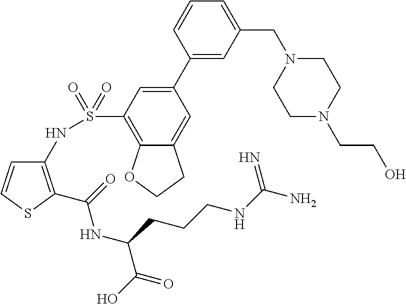 |
| Yield | White solid, 74 mg, 66% |
| LC-MS | Rₜ 4.42 min.; purity 100%; MS m/z − 700 [M + 1]⁺. |

### General Procedure for Amino-Thiazole Aldehyde Formation

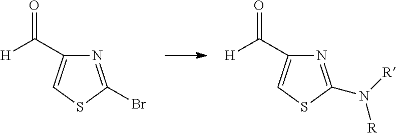

The bromo-thiazole (1 eq), amine (commercially available; 1.1 eq) and lithium hydroxide (1.15 eq) were combined and dissolved in tetrahydrofuran (2 mL)/water (0.1 mL). The reaction mixtures were heated in the microwave at 75° C. for 15 minutes. After this time water (5 mL) was added to the reaction mixtures and the pH adjusted to approx 7 using hydrochloric acid (1M, aqueous solution). The solvents were removed in vacuo, re-dissolved in dimethylsulfoxide and either used without further purification or purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% Table 5 summarises the thiazole aldehydes constructed using this method.

TABLE 5

| | |
|---|---|
| ID | 2-(4-Pyridin-4-yl-piperazin-1-yl)-thiazole-4-carbaldehyde |
| Structure | 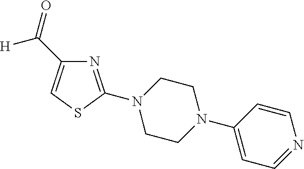 |
| Yield | Brown oil. 9 mg, 8%. |
| LC-MS | Rₜ 1.21 min.; purity 92%; MS m/z - 275 [M + 1]⁺. |
| ID | 4-(4-Formyl-thiazol-2-yl)-piperazine-1-carboxylic acid tert- |
| | butyl ester |
| Structure | 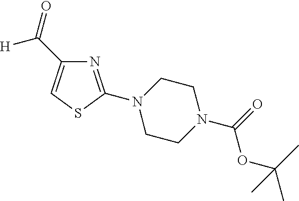 |
| Yield | Yellow oil. 29 mg, 23%. |
| LC-MS | Rₜ 3.76 min.; purity 100%; MS m/z - 298 [M + 1]⁺. |
| ID | 2-(4-Pyridin-3-ylmethyl-piperazin-1-yl)-thiazole-4- |
| | carbaldehyde |
| Structure | 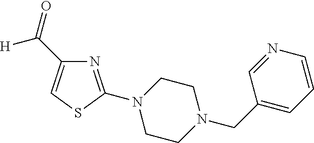 |
| Yield | Dark brown oil. Used without preparative LC-MS purification. |
| LC-MS | Rₜ 1.06 min.; purity 80%; MS m/z - 289 [M + 1]⁺. |
| ID | 2-(4-Morpholin-4-yl-piperadin-1-yl)-thiazole-4-carbaldehyde |
| Structure | 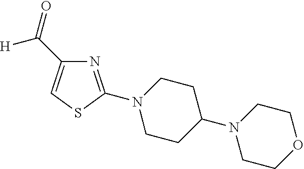 |
| Yield | Brown oil. Used without preparative LC-MS purification. |
| LC-MS | Rₜ 0.55 min.; purity 75%; MS m/z - 282 [M + 1]⁺. |
| ID | 2-(4-Pyridin-4-ylmethyl-piperazin-1-yl)-thiazole-4- |
| | carbaldehyde |
| Structure | 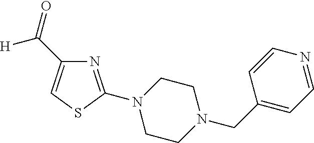 |
| Yield | Yellow oil. Used without preparative LC-MS purification. |
| LC-MS | Rₜ 1.19 min.; purity 60%; MS m/z - 289 [M + 1]⁺. |
| ID | 2-(4-Pyridin-3-yl-piperazin-1-yl)-thiazole-4-carbaldehyde |
| Structure | 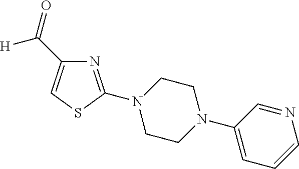 |
| Yield | Colourless oil. 21 mg, 18%. |
| LC-MS | Rₜ 1.17 + 1.26 min.; purity 85%; MS m/z - 275 [M + 1]⁺. |
| ID | 2-(4-Pyridin-2-yl-piperazin-1-yl)-thiazole-4-carbaldehyde |
| Structure | 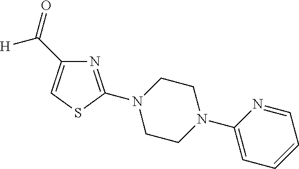 |
| Yield | White solid. 46 mg, 43%. |
| LC-MS | Rₜ 1.48 min.; purity 100%; MS m/z - 275 [M + 1]⁺. |
| ID | [1-(4-Formyl-thiazol-2-yl)-piperidin-4-yl]-carbamic acid tert- |
| | butyl ester |
| Structure | 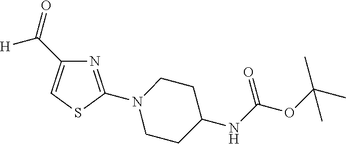 |
| Yield | Brown solid. Used without preparative LC-MS purification. |
| LC-MS | Rₜ 3.69 min.; purity 85%; MS m/z - 312 [M + 1]⁺. |
| ID | 2-(4-Pyridin-2-ylmethyl-piperazin-1-yl)-thiazole-4- |
| | carbaldehyde |
| Structure | 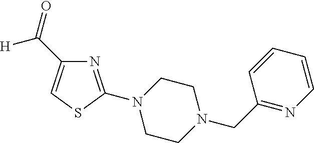 |
| Yield | Brown oil. Used without preparative LC-MS purification. |
| LC-MS | Rₜ 1.16 min.; purity 81%; MS m/z - 289 [M + 1]⁺. |
| ID | 2-(4-Pyrimidin-2-yl-piperazin-1-yl)-thiazole-4-carbaldehyde |
| Structure | 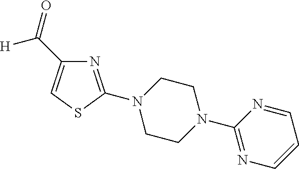 |
| Yield | Pale yellow solid. 12 mg, 11%. |
| LC-MS | Rₜ 3.21 min.; purity 95%; MS m/z - 276 [M + 1]⁺. |
| ID | 2-[(5-Methyl-isoxazol-3-ylmethyl)-amino]-thiazole-4- |
| | carbaldehyde |
| Structure | 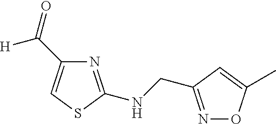 |
| Yield | Pale yellow solid. 10 mg, 12%. |
| LC-MS | Rₜ 1.97 min.; purity 95%; MS m/z - 224 [M + 1]⁺. |
| ID | 2-(2,3,5,6-Tetrahydro-[1,2′]bipyrazinyl-4-yl)-thiazole-4- |
| | carbaldehyde |
| Structure | 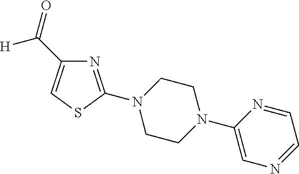 |
| Yield | Yellow solid. 23 mg, 21%. |
| LC-MS | Rₜ 3.04 min.; purity 80%; MS m/z - 276 [M + 1]⁺. |
| ID | 2-(4-[1,3,5]Triazin-2-yl-piperazin-1-yl)-thiazole-4- |
| | carbaldehyde |
| Structure | 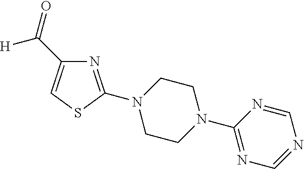 |
| Yield | Yellow solid. 11 mg, 10%. |
| LC-MS | Rₜ 2.68 min.; purity >95%; MS m/z - 277 [M + 1]⁺. |
| ID | 2-(4-Thiazol-2-yl-piperazin-1-yl)-thiazole-4-carbaldehyde |
| Structure | 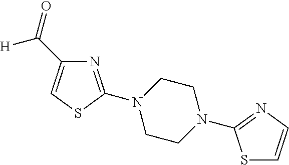 |
| Yield | Brown oil. 36 mg, 33%. |
| LC-MS | Rₜ 2.52 min.; purity 75%; MS m/z - 281 [M + 1]⁺. |
| ID | 2-(Ethyl-pyridin-4-ylmethyl-amino)-thiazole-4-carbaldehyde |
| Structure | 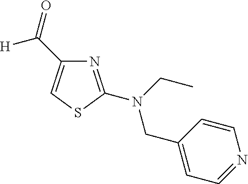 |
| Yield | Yellow oil. 14 mg, 14%. |
| LC-MS | Rₜ 1.02 min.; purity 83%; MS m/z - 248 [M + 1]⁺. |

### General Procedure for Solution-Phase Reductive Amination

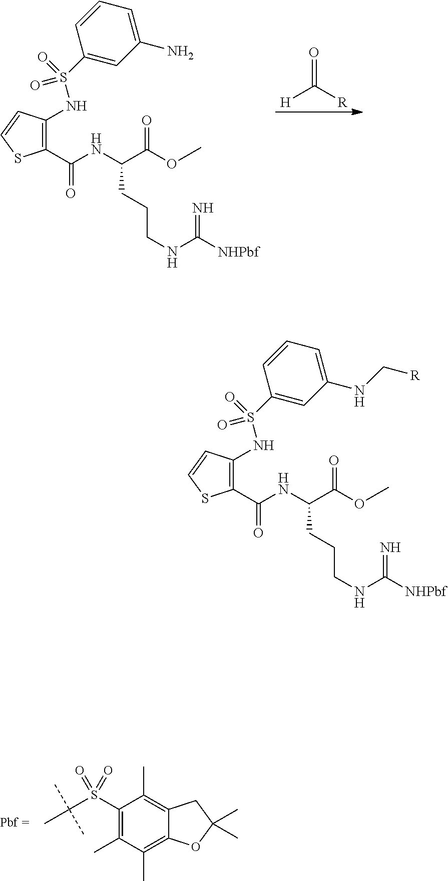

The aniline (1 eq), aldehyde (either commercially available or prepared as above; 0.5-6.0 eq) and sodium cyanoborohydride (0.6-2.0 eq) were combined and dissolved in methanol (2 mL). Hydrochloric acid (1M, aqueous solution) or acetic acid was then added until a pH of between 5-6 was reached and the reaction mixtures were stirred at 20° C. for 16 hours. The solvents were removed in vacuo and the resulting residues were re-dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/methanol. The purified peptidomimetics were isolated via solvent evaporation.

Table 6 summarises the compounds constructed using this method.

TABLE 6

| | |
|---|---|
| ID | (S)-5-guanidino(Pbf)-2-[(3-{3-[2-(4-pyridin-4-yl-piperazin-1- |
| | yl)-thiazol-4-ylmethyl]-amino}-benzenesulfonylamino)- |
| | thiophene-2-carbonyl]-amino}-pentanoic acid methyl ester |
| Struc- ture | 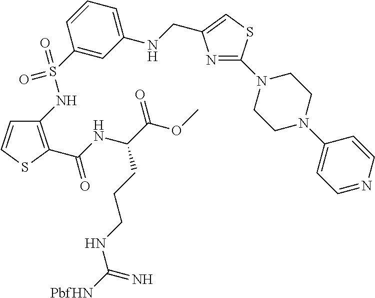 |
| Yield | Clear oil. 9.0 mg, 28%. |
| LC-MS | Rₜ 3.53 min.; purity >95%; MS m/z - 979 [M + 1]⁺. |
| ID | (S)-4-[4-({3-[2-(4-guanidino(Pbf)-1-methoxycarbonyl- |
| | butylcarbamoyl)-thiophene-3-ylsulfamoyl]-phenylamino}- |
| | methyl)-thiazol-2-yl]-piperazine-1-carboxylic acid tert-butyl |
| | ester |
| Struc- ture | 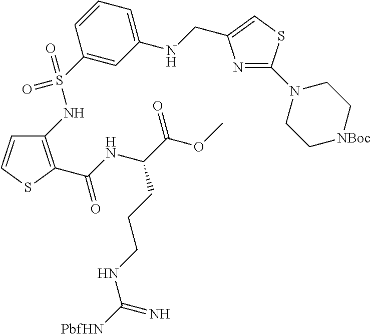 |
| Yield | White solid. 22 mg, 22%. |
| LC-MS | Rₜ 4.82 min.; purity >90%; MS m/z - 1002 [M + 1]⁺. |
| ID | (S)-5-guanidino(Pbf)-2-[(3-{3-[2-(4-pyridin-3-ylmethyl- |
| | piperazin-1-yl)-thiazol-4-ylmethyl]-amino}- |
| | benzenesulfonylamino)-thiophene-2-carbonyl]-amino}- |
| | pentanoic acid methyl ester |
| Struc- ture | 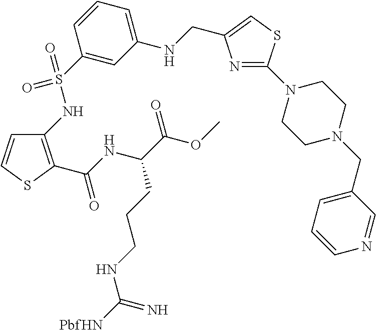 |
| Yield | White solid. 27 mg, 19%. |
| LC-MS | Rₜ 3.94 min.; purity >95%; MS m/z - 993 [M + 1]⁺. |
| ID | (S)-5-guanidino(Pbf)-2-[(3-{3-[2-(4-morpholin-4-yl-piperidin-1- |
| | yl)-thiazol-4-ylmethyl]-amino}-benzenesulfonylamino)- |
| | thiophene-2-carbonyl]-amino}-pentanoic acid methyl ester |
| Struc- ture | 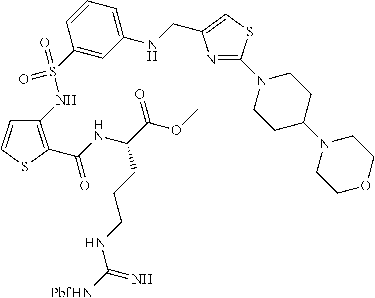 |
| Yield | White solid. 22 mg, 16%. |
| LC-MS | Rₜ 3.52 min.; purity >95%; MS m/z - 986 [M + 1]⁺. |
| ID | (S)-5-guanidino(Pbf)-2-[(3-{3-[2-(4-pyridin-4-ylmethyl- |
| | piperazin-1-yl)-thiazol-4-ylmethyl]-amino}- |
| | benzenesulfonylamino)-thiophene-2-carbonyl]-amino}- |
| | pentanoic acid methyl ester |
| Struc- ture | 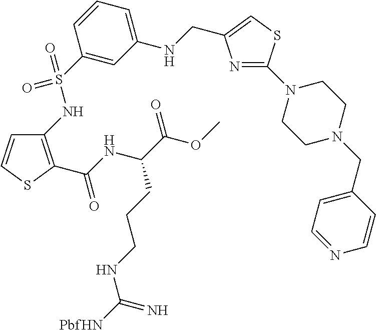 |
| Yield | Off-white solid. 5.0 mg, 5%. |
| LC-MS | Rₜ 4.05 min.; purity >90%; MS m/z - 993 [M + 1]⁺. |
| ID | (S)-5-guanidino(Pbf)-2-[(3-{3-[2-(4-pyridin-3-yl-piperazin-1- |
| | yl)-thiazol-4-ylmethyl]-amino}-benzenesulfonylamino)- |
| | thiophene-2-carbonyl]-amino}-pentanoic acid methyl ester |
| Struc- ture | 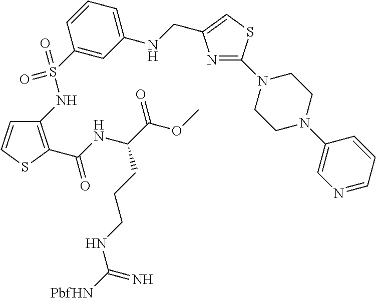 |
| Yield | Clear oil. 24 mg, 32%. |
| LC-MS | Rₜ 3.89 min.; purity >95%; MS m/z - 979 [M + 1]⁺. |
| ID | (S)-5-guanidino(Pbf)-2-[(3-{3-[2-(4-pyridin-2-yl-piperazin-1- |
| | yl)-thiazol-4-ylmethyl]-amino}-benzenesulfonylamino)- |
| | thiophene-2-carbonyl]-amino}-pentanoic acid methyl ester |
| Struc- ture | 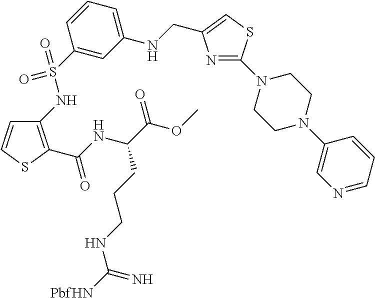 |
| Yield | Clear oil. 42 mg, 29%. |
| LC-MS | Rₜ 4.26 min.; purity >95%; MS m/z - 979 [M + 1]⁺. |
| ID | (S)-2-[(3-{3-[2-(4-tert-Butoxycarbonylamino-piperidin-1-yl)- |
| | thiazol-4-ylmethyl]-amino}-benzenesulfonylamino)-thiophene- |
| | 2-carbonyl]-amino}-5-guanidino-(Pbf)-pentanoic acid methyl |
| | ester |
| Struc- ture | 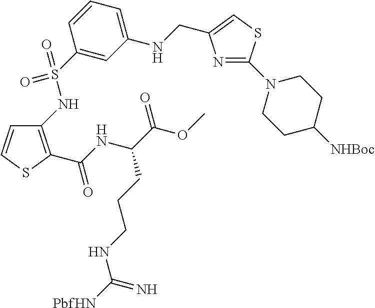 |
| Yield | Pale brown oil. 34 mg, 22%. |
| LC-MS | Rₜ 4.71 min.; purity >95%; MS m/z - 1016 [M + 1]⁺. |
| ID | (S)-5-guanidino(Pbf)-2-[(3-{3-[2-(4-pyridin-2-ylmethyl- |
| | piperazin-1-yl)-thiazol-4-ylmethyl]-amino}- |
| | benzenesulfonylamino)-thiophene-2-carbonyl]-amino}- |
| | pentanoic acid methyl ester |
| Struc- ture | 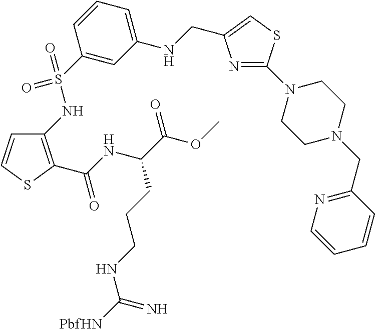 |
| Yield | Clear oil. 16 mg, 11%. |
| LC-MS | Rₜ 3.82 min.; purity >95%; MS m/z - 993 [M + 1]⁺. |
| ID | (S)-5-guanidino(Pbf)-2-[(3-{3-[2-(4-pyrimidin-2-yl-piperazin-1- |
| | yl)-thiazol-4-ylmethyl]-amino}-benzenesulfonylamino)- |
| | thiophene-2-carbonyl]-amino}-pentanoic acid methyl ester |
| Struc- ture | 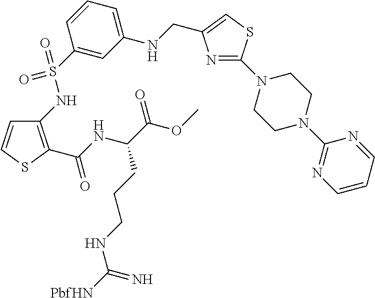 |
| Yield | White solid. 11 mg, 28% |
| LC-MS | Rₜ 4.71 min.; purity >90%; MS m/z - 980 [M + 1]⁺. |
| ID | (S)-5-guanidino(Pbf)-2-({3-[3-({2-[(5-methyl-isoxazol-3- |
| | ylmethyl)-amino]-thiazol-4-ylmethyl}-amino)- |
| | benzenesulfonylamino]-thiophene-2-carbonyl}-amino)- |
| | pentanoic acid methyl ester |
| Struc- ture | 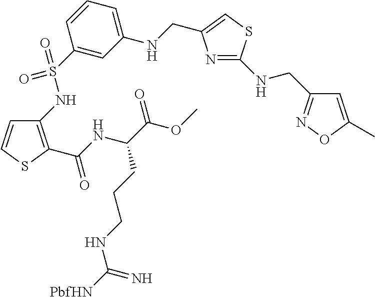 |
| Yield | White solid. 15 mg, 40%. |
| LC-MS | Rₜ 4.41 min.; purity >75%; MS m/z - 928 [M + 1]⁺. |
| ID | (S)-5-Guanidino(Pbf)-2-[(3-{3-[(2-morpholin-4-yl-thiazol-4- |
| | ylmethyl)-amino]-benzenesulfonylamino}-thiophene-2- |
| | carbonyl)-amino]-pentanoic acid methyl ester |
| Struc- ture | 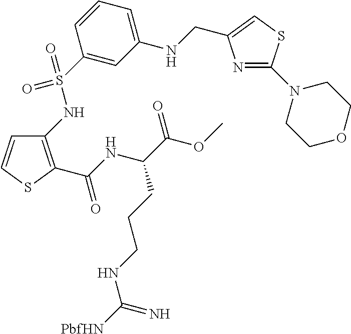 |
| Yield | Clear oil. 18 mg, 14%. |
| LC-MS | Rₜ 4.51 min.; purity 67%; MS m/z - 903 [M + 1]⁺. |
| ID | (S)-5-Guanidino(Pbf)-2-{[3-(3-{[2-(4-[1,3,5]triazin-2-yl- |
| | piperazin-1-yl)-thiazol-4-ylmethyl]-amino}- |
| | benzenesulfonylamino)-thiophene-2-carbonyl]-amino}- |
| | pentanoic acid methyl ester |
| Struc- ture | 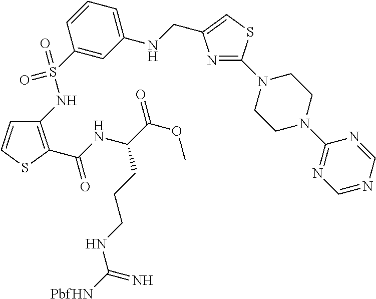 |
| Yield | White solid. 2.0 mg, 5%. |
| LC-MS | Rₜ 4.58 min.; purity >75%; MS m/z - 981 [M + 1]⁺. |
| ID | (S)-5-guanidino(Pbf)-2-{[3-(3-{[2-(4-thiazol-2-yl-piperazin-1- |
| | yl)-thiazol-4-ylmethyl]-amino}-benzenesulfonylamino)- |
| | thiophene-2-carbonyl]-amino}-pentanoic acid methyl ester |
| Struc- ture | 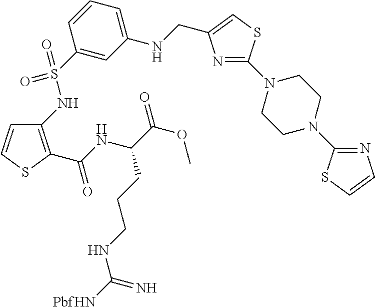 |
| Yield | White solid. 5.0 mg, 4%. |
| LC-MS | Rₜ 4.63 min.; purity >75%; MS m/z - 985 [M + 1]⁺. |
| ID | (S)-2-{[3-(3-{[2-(Ethyl-pyridin-4-ylmethyl-amino)-thiazol-4- |
| | ylmethyl]-amino}-benzenesulfonylamino)-thiophene-2- |
| | carbonyl]-amino}-5-guanidino(Pbf)-pentanoic acid methyl ester |
| Struc- ture | 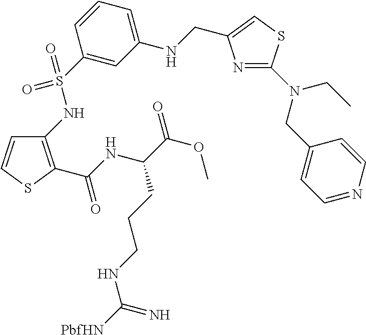 |
| Yield | Clear oil. 7.0 mg, 13%. |
| LC-MS | Rₜ 2.88 min.; purity 62%; MS m/z - 952 [M + 1]⁺. |
| ID | (S)-2-[(3-{3-[(2-Bromo-thiazol-4-ylmethyl)-amino]- |
| | benzenesulfonylamino}-thiophene-2-carbonyl)-amino]-5- |
| | guanidino(Pbf)-pentanoic acid methyl ester. |
| Struc- ture | 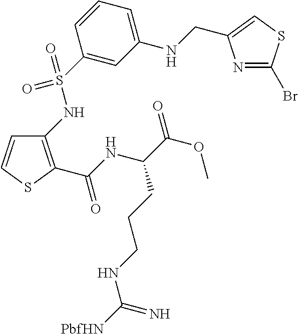 |
| Yield | White solid, 3.31 g, 45% |
| LC-MS | Rₜ 3.19 min.; purity 99%; MS m/z - 896/898 [M + 1]⁺. |

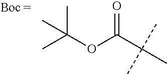

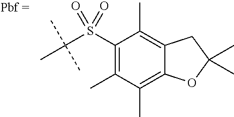

### General Procedure for Aminoethyl-Pyrazole Aldehyde Formation and Subsequent Reductive Amination

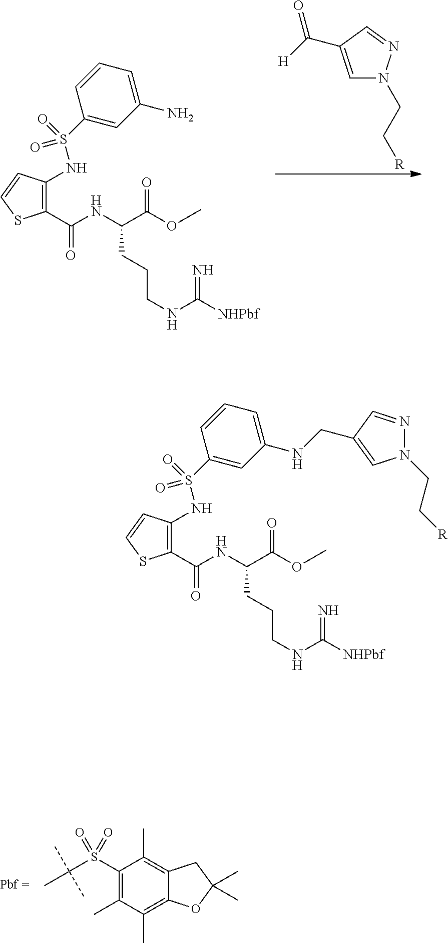

1-(2-chloroethyl)-1H-pyrazole-4-carbaldehyde (4.2 eq), amine (5 eq) and triethylamine (8.4 eq) were heated together in N-methyl-2-pyrrolidone (1 mL) at 85° C. for 16 hours. The solvents were removed in vacuo and aniline (1 eq), in methanol (1 mL), and sodium cyanoborohydride (2.0 eq) were added. Hydrochloric acid (1M, aqueous solution) was then added until a pH of between 5-6 was reached and the reaction mixtures were stirred at 20° C. for 16 hours. The methanol was removed in vacuo and the mixtures diluted with dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/methanol. The purified peptidomimetics were isolated via solvent evaporation and used without further purification.

Table 7 summarises the compounds constructed using this method.

TABLE 7

| | |
|---|---|
| ID | (S)-5-guanidino(Pbf)-2-({3-[3-({1-(4-pyridin-2-yl-piperazin-1- |
| | yl)-ethyl]-1H-pyrazol-4-ylmethyl}-amino)- |
| | benzenesulfonylamino]-thiophene-2-carbonyl}-amino)- |
| | pentanoic acid methyl ester |
| Struc- ture | 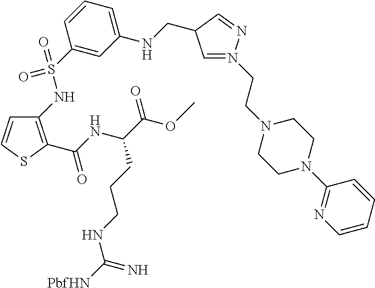 |
| Yield | Pale yellow oil. 16 mg, 11%. |
| LC-MS | Rₜ 3.03 min.; purity >90%; MS m/z - 865 [M + 1]⁺. |

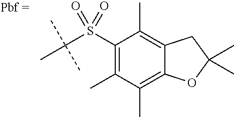

General Procedure for Displacement Reaction with Bromo-Thiazole

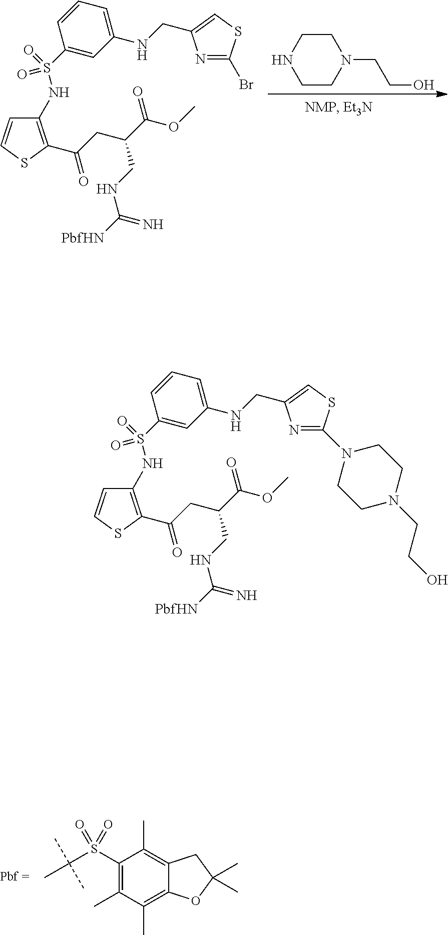

The bromo-thiazole (1 eq) and 1-(2-hydroxyethyl)piperazine (10 eq) were combined and dissolved in triethylamine (10 eq) and N-methyl-2-pyrrolidone (2 mL) before being heated at reflux for 24 hours. The mixture was cooled to ambient temperature and directly purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/methanol. The purified peptidomimetics were isolated via solvent evaporation.

Table 8 (below) summarises the compounds constructed using this method.

| | |
|---|---|
| ID | (S)-5-guanidino(Pbf)-2-({3-({2-[4-(2-hydroxy-ethyl)-piperazin-1- |
| | yl]-thiazol-4-ylmethyl}-amino)-benzenesulfonylamino]-thiophene- |
| | 2-carbonyl}-amino)-pentanoic acid methyl ester. |
| Struc- ture | 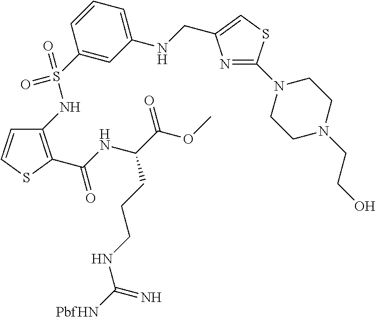 |
| Yield | White solid, 96 mg, 36% |
| LC-MS | Rₜ 2.31 min.; purity 87%; MS m/z - 946 [M + 1]⁺. |

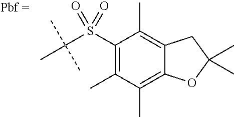

### General Procedure for Solution-Phase Alkylation

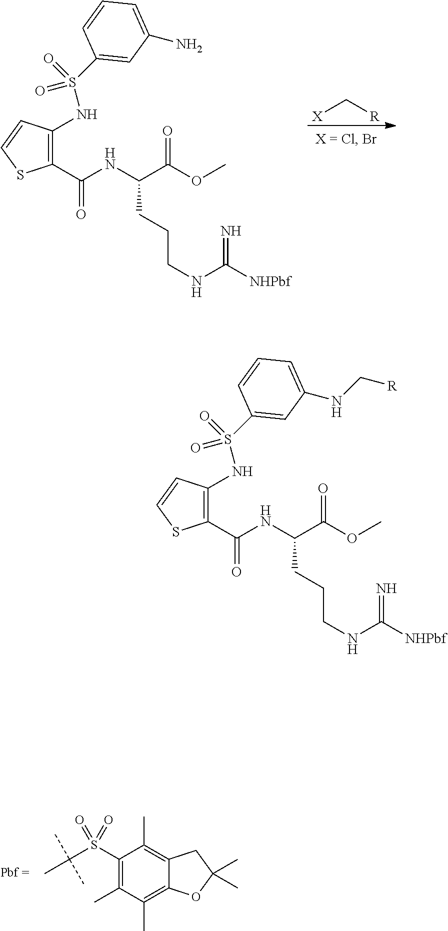

The aniline (1 eq) was dissolved in methanol (2 mL) or dimethylsulfoxide (2 mL). The alkyl halide (1 eq) was added, followed by triethylamine (2 eq) and the reaction mixtures were stirred at 50-100° C. for 16 hours. If methanol was used as the solvent it was removed in vacuo and the resulting residues were re-dissolved in dimethylsulfoxide and purified by (mass-directed) preparative LC-MS using a preparative C-18 column (Phenomenex Luna C18 (2), 100×21.2 mm, 5 μM) and a linear AB gradient of 5-95% for B over 12 min at a flow rate of 20 mL/minute, where eluent A was 0.1% formic acid/water and eluent B was 0.1% formic acid/methanol. The purified peptidomimetics were isolated via solvent evaporation and used without further purification

Table 9 summarises the compounds constructed using this method.

TABLE 9

| | |
|---|---|
| ID | (S)-5-Guanidino(Pbf)-2-{[3-(3-{[5-(2-methyoxy-phenyl)- |
| | [1,2,4]oxadiazol-3-ylmethyl]-amino}-benzenesulfonylamino)- |
| | thiophene-2-carbonyl]-amino}-pentanoic acid methyl ester |
| Struc- ture | 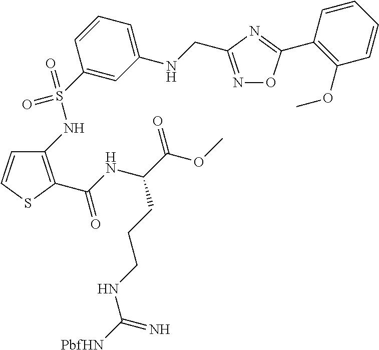 |
| Yield | Clear oil. 8.0 mg, 6%. |
| LC-MS | Rₜ 4.64 min.; purity >80%; MS m/z - 909 [M + 1]⁺. |
| ID | (S)-4-({4-[2-((S)-4-Guanidino(Pbf)-1-methoxycarbonyl- |
| | butylcarbamoyl)-thiophen-3-ylsulfamoyl]-phenylamino}-methyl)- |
| | thiazole-2-carboxylic acid ethyl ester |
| Struc- ture | 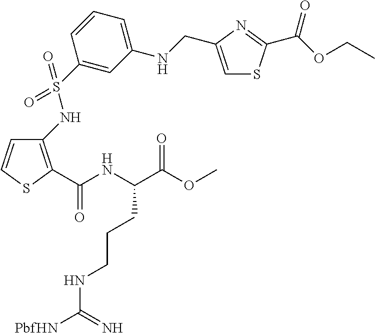 |
| Yield | Clear oil. 6.5 mg, 6%. |
| LC-MS | Rₜ 4.53 min.; purity >95%; MS m/z - 890 [M + 1]⁺. |
| ID | (S)-2-{[3-(3-{[(3-Cyano-phenylcarbamoyl)-methyl]-amino}- |
| | benzenesulfonylamino)-thiophene-2-carbonyl]-amino}-5- |
| | guanidino(Pbf)-pentanoic acid methyl ester |
| Struc- ture | 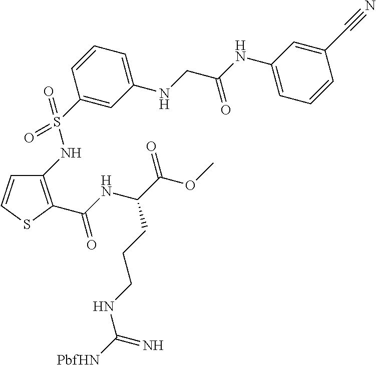 |
| Yield | Clear oil. 17 mg, 13%. |
| LC-MS | Rₜ 4.47 min.; purity 80%; MS m/z - 879 [M + 1]⁺. |

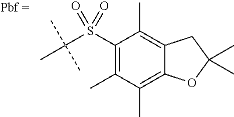

### General Procedure for Methyl Ester Hydrolysis and Pbf/Boc Removal

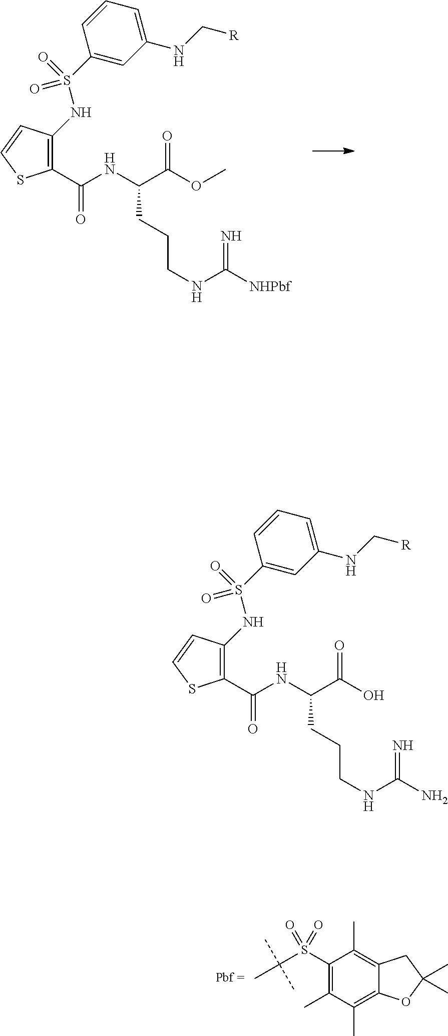

The fully protected starting materials (1 eq) were stirred with lithium hydroxide (5 eq) in tetrahydrofuran/water (4:1; 2.5 mL) at 20-60° C. for 1-3 hours, as necessary. After this time the solvents were removed in vacuo, and the residues treated with trifluoroacetic acid (2 mL) and water (0.1 mL). The reaction mixtures were stirred at 20° C. for a further 3-16 hours. The solvents were removed in vacuo and the resulting residues were re-dissolved in dimethylsulfoxide and purified by (mass-directed) preparative LC-MS using a preparative C-18 column (Phenomenex Luna C18 (2), 100×21.2 mm, 5 μM) and a linear AB gradient of 2-95% for B over 12 min at a flow rate of 20 mL/minute, where i., eluent A was 0.1% formic acid/water and eluent B was 0.1% formic acid/acetonitrile or ii., eluent A was 10 mM ammonium bicarbonate (pH9) and eluent B was neat methanol. The purified peptidomimetics were isolated via solvent evaporation.

Table 10 summarises the compounds constructed using this method.

TABLE 10

| | |
|---|---|
| ID | 56; (S)-5-guanidino-2-[(3-{3-[(2-piperidin-4-yl-thiazol-4- |
| | ylmethyl)-amino]-benzenesulfonylamino}-thiophene-2- |
| | carbonyl)-amino]-pentanoic acid |
| Struc- ture | 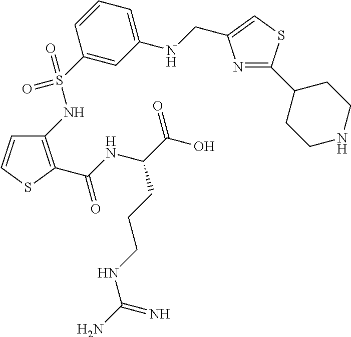 |
| Yield | Off white solid, 4.4 gm, 78% |
| LC-MS | Rₜ 4.24 min.; purity 99%; MS m/z - 635 [M + 1]⁺. |
| ID | 57; (S)-5-guanidino-2-{[3-(3-{[2-(4-pyridin-4-yl-piperazin-1- |
| | yl)-thiazol-4-ylmethyl]-amino}-benzenesulfonylamino)- |
| | thiophene-2-carbonyl]-amino}-pentanoic acid |
| Struc- ture | 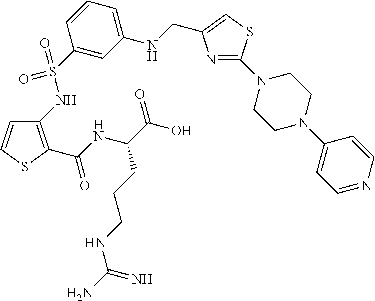 |
| Yield | Brown oil, 1.9 mg, 27% |
| LC-MS | Rₜ 4.47 min.; purity 88%; MS m/z - 713 [M + 1]⁺. |
| ID | 58; (S)-5-guanidino-2-[(3-{3-[(2-piperazin-1-yl-thiazol-4- |
| | ylmethyl)-amino]-benzenesulfonylamino}-thiophene-2- |
| | carbonyl]-amino]-pentanoic acid |
| Struc- ture | 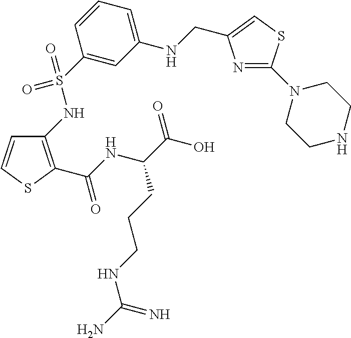 |
| Yield | Brown oil, 11.8 mg, 60% |
| LC-MS | Rₜ 4.2 min.; purity 95%; MS m/z - 636 [M + 1]⁺. |
| ID | 59; (S)-5-guanidino-2-{[3-(3-{[2-(4-pyridin-3-ylmethyl- |
| | piperazin-1-yl)-thiazol-4-ylmethyl]-amino}- |
| | benzenesulfonylamino)-thiophene-2-carbonyl]-amino}- |
| | pentanoic acid |
| Struc- ture | 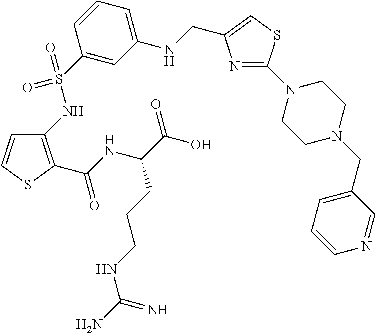 |
| Yield | Brown oil, 9 mg, 40% |
| LC-MS | Rₜ 4.60 min.; purity 99%; MS m/z - 727 [M + 1]⁺. |
| ID | 60; (S)-5-guanidino-2-{[3-(3-{[2-(4-morpholin-4-yl-piperidin- |
| | 1-yl)-thiazol-4-ylmethyl]-amino}-benzenesulfonylamino)- |
| | thiophene-2-carbonyl]-amino}-pentanoic acid |
| Struc- ture | 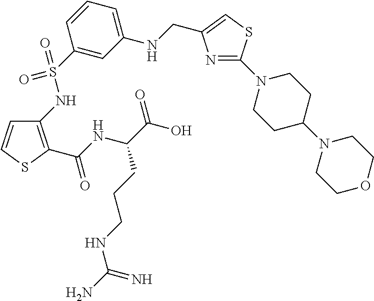 |
| Yield | Brown oil, 9.5 mg, 40% |
| LC-MS | Rₜ 4.29 min.; purity 93%; MS m/z - 720 [M + 1]⁺. |
| ID | 61; (S)-5-guanidino-2-{[3-(3-{[2-(4-pyridin-4-ylmethyl- |
| | piperazin-1-yl)-thiazol-4-ylmethyl]-amino}- |
| | benzenesulfonylamino)-thiophene-2-carbonyl]-amino}- |
| | pentanoic acid |
| Struc- ture | 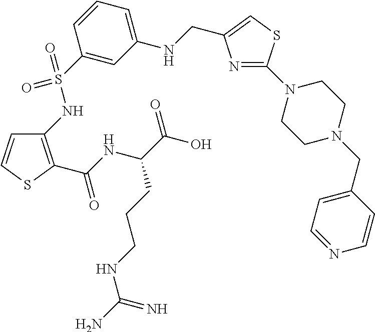 |
| Yield | White solid, 1.3 mg, 32% |
| LC-MS | Rₜ 4.71 min.; purity 93%; MS m/z - 727 [M + 1]⁺. |
| ID | 62; (S)-5-guanidino-2-{[3-(3-{[2-(4-pyridin-3-yl-piperazin-1- |
| | yl)-thiazol-4-ylmethyl]-amino}-benzenesulfonylamino)- |
| | thiophene-2-carbonyl]-amino}-pentanoic acid |
| Struc- ture | 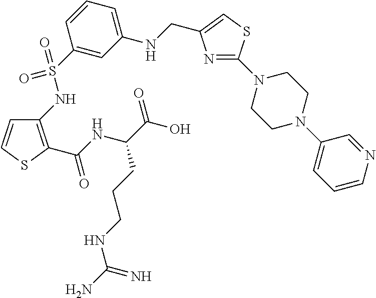 |
| Yield | Pale yellow oil, 10.8 mg, 57% |
| LC-MS | Rₜ 4.63 min.; purity 97%; MS m/z - 713 [M + 1]⁺. |
| ID | 63; (S)-5-guanidino-2-{[3-(3-{[2-(4-pyridin-2-yl-piperazin-1- |
| | yl)-thiazol-4-ylmethyl]-amino}-benzenesulfonylamino)- |
| | thiophene-2-carbonyl]-amino}-pentanoic acid |
| Struc- ture | 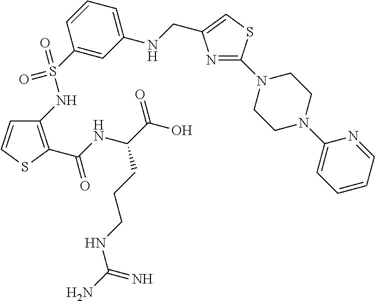 |
| Yield | Colouless oil, 23 mg, 70% |
| LC-MS | Rₜ 5.02 min.; purity 95%; MS m/z - 713 [M + 1]⁺. |
| ID | 64; (S)-5-guanidino-2-{[3-(3-{[2-(4-amino-piperidin-1-yl)- |
| | thiazol-4-ylmethyl]-amino}-benzenesulfonylamino)-thiophene- |
| | 2-carbonyl]-amino}-pentanoic acid |
| Struc- ture | 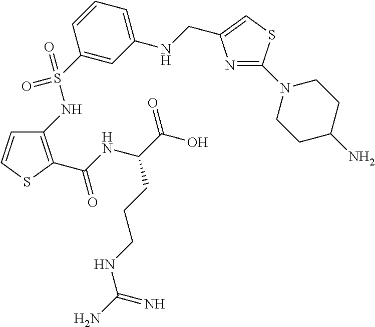 |
| Yield | Pale brown oil, 16.1 mg, 70% |
| LC-MS | Rₜ 4.16 min.; purity 97%; MS m/z - 650 [M + 1]⁺. |
| ID | 65; (S)-5-guanidino-2-{[3-(3-{[2-(4-pyridin-2-ylmethyl- |
| | piperazin-1-yl)-thiazol-4-ylmethyl]-amino}- |
| | benzenesulfonylamino)-thiophene-2-carbonyl]-amino}- |
| | pentanoic acid |
| Struc- ture | 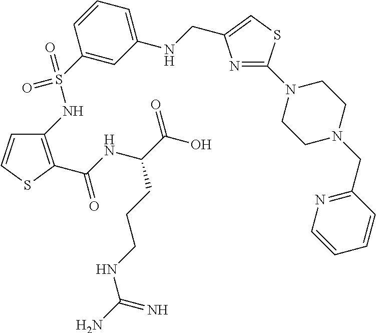 |
| Yield | Colourless oil, 10.3 mg, 83% |
| LC-MS | Rₜ 4.57 min.; purity 95%; MS m/z - 727 [M + 1]⁺. |
| ID | 66; (S)-5-guanidino-2-{[3-(3-{[2-(4-pyrimidin-2-yl-piperazin- |
| | 1-yl)-thiazol-4-ylmethyl]-amino}-benzenesulfonylamino)- |
| | thiophene-2-carbonyl]-amino}-pentanoic acid |
| Struc- ture | 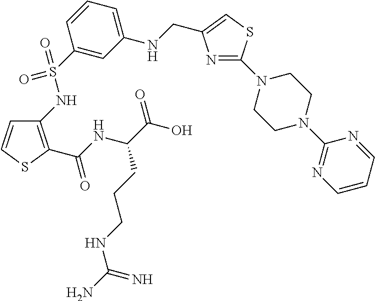 |
| Yield | Beige solid, 4.6 mg, 58% |
| LC-MS | Rₜ 6.23 min.; purity 95%; MS m/z - 714 [M + 1]⁺. |
| ID | 67; (S)-5-guanidino-2-({3-[3-({2-[(methyl-isoxazol-3- |
| | ylmethyl)-amino]-benzenesulfonylamino]-thiophene-2- |
| | carbonyl}-amino)-pentanoic acid |
| Struc- ture | 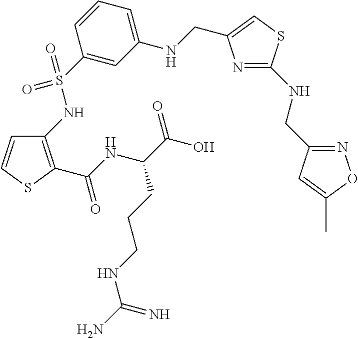 |
| Yield | Yellow solid, 7 mg, 66% |
| LC-MS | Rₜ 5.47 min.; purity 84%; MS m/z - 6623 [M + 1]⁺. |
| ID | 68; (S)-5-guanidino-2-({3-[3-({1-(4-pyridin-2-yl-piperazin-1- |
| | yl)-ethyl]-1H-pyrazol-4-ylmethyl}-amino)- |
| | benzenesulfonylamino]-thiophene-2-carbonyl}-amino)- |
| | pentanoic acid |
| Struc- ture | 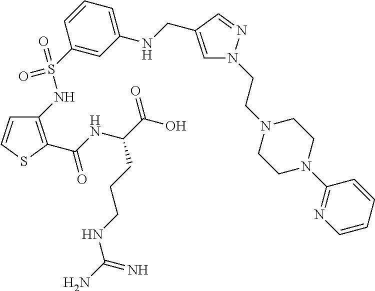 |
| Yield | Beige solid, 8.6 mg, 69% |
| LC-MS | Rₜ 4.38 min.; purity 96%; MS m/z - 724 [M + 1]⁺. |
| ID | 69; (S)-5-Guanidino-2-{[3-(3-{[5-(2-methyoxy-phenyl)- |
| | [1,2,4]oxadiazol-3-ylmethyl]-amino}-benzenesulfonylamino)- |
| | thiophene-2-carbonyl]-amino}-pentanoic acid |
| Struc- ture | 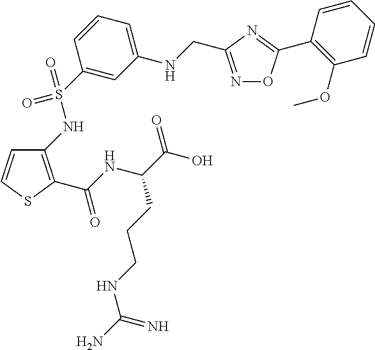 |
| Yield | Yellow solid, 3.3 mg, 40% |
| LC-MS | Rₜ 6.13 min.; purity 80%; MS m/z - 643 [M + 1]⁺. |
| ID | 70; (S)-2-{[3-(3-{[(3-Cyano-phenylcarbamoyl)-methyl]- |
| | amino}-benzenesulfonylamino)-thiophene-2-carbonyl]-amino}- |
| | 5-guanidino-pentanoic acid |
| Struc- ture | 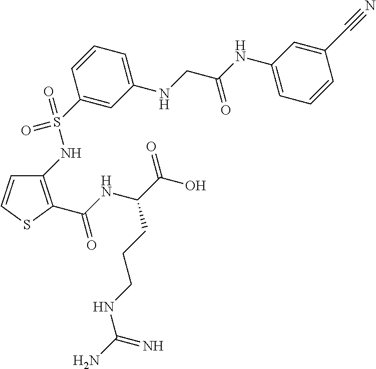 |
| Yield | White solid, 2 mg, 16% |
| LC-MS | Rₜ 5.62 min.; purity 88%; MS m/z - 613 [M + 1]⁺. |
| ID | 71; (S)-5-guanidino-2-[(3-{3-[(2-morpholin-4-yl-thiazol-4- |
| | ylmethyl)-amino]-benzenesulfonylamino}-thiophene-2- |
| | carbonyl]-amino]-pentanoic acid |
| Struc- ture | 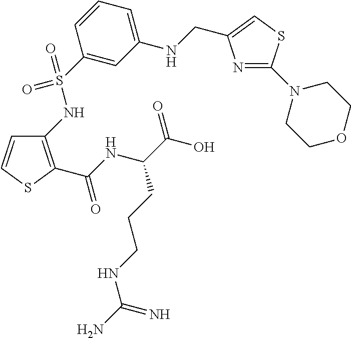 |
| Yield | White solid, 4.1 mg, 30% |
| LC-MS | Rₜ 5.62 min.; purity 94%; MS m/z - 637 [M + 1]⁺. |
| ID | 72; (S)-4-({3-[2-1-Carboxyl-4-guanidino-butylcarbamoyl)- |
| | thiophen-3-yl sulfamoyl]-phenylamino}-methyl)-thiazole-2- |
| | carboxylic acid |
| Struc- ture | 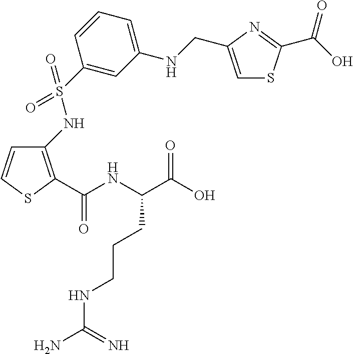 |
| Yield | Beige solid, 1.4 mg, 29% |
| LC-MS | Rₜ 5.65 min.; purity 77%; MS m/z - 596 [M + 1]⁺. |
| ID | 73; (S)-2-{[3-(3-{[2-(Ethyl-pyridin-4-ylmethyl-amino)-thiazol- |
| | 4-ylmethyl]-amino}-benzenesulfonylamino)-thiophene-2- |
| | carbonyl]-amino}-5-guanidino-pentanoic acid |
| Struc- ture | 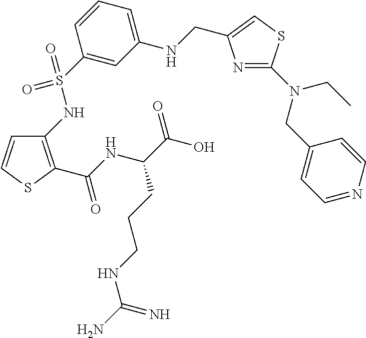 |
| Yield | White solid, 1.6 mg, 30% |
| LC-MS | Rₜ 4.98 min.; purity 93%; MS m/z - 686 [M + 1]⁺. |
| ID | 74; (S)-5-Guanidino-2-{[3-(3-{[2-(2,3,5,6-tetrahydro- |
| | [1,2′]bipyrazinyl-4-ylmethyl]-amino}-benzenesulfonylamino)- |
| | thiophene-2-carbonyl]-amino}-pentanoic acid |
| Struc- ture | 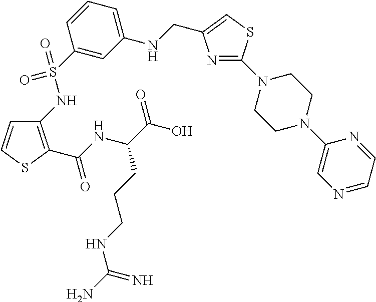 |
| Yield | Off white solid, 3.2 mg, 29% |
| LC-MS | Rₜ 6.06 min.; purity 96%; MS m/z - 714 [M + 1]⁺. |
| ID | 75; (S)-5-guanidino-2-{[3-(3-{[2-(4-[1,3,5]triazin-2-yl- |
| | piperazin-1-yl)-thiazol-4-ylmethyl]-amino}- |
| | benzenesulfonylamino)-thiophene-2-carbonyl]-amino}- |
| | pentanoic acid |
| Struc- ture | 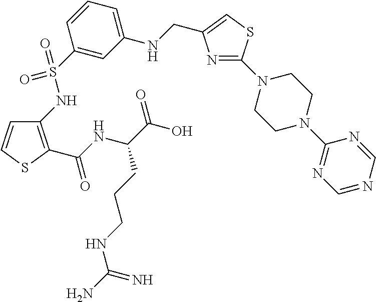 |
| Yield | White solid, 0.9 mg, 60% |
| LC-MS | Rₜ 5.92 min.; purity 98%; MS m/z - 715 [M + 1]⁺. |
| ID | 76; (S)-5-guanidino-2-{[3-(3-{[2-(4-thiazol-2-yl-piperazin-1- |
| | yl)-thiazol-4-ylmethyl]-amino}-benzenesulfonylamino)- |
| | thiophene-2-carbonyl]-amino}-pentanoic acid |
| Struc- ture | 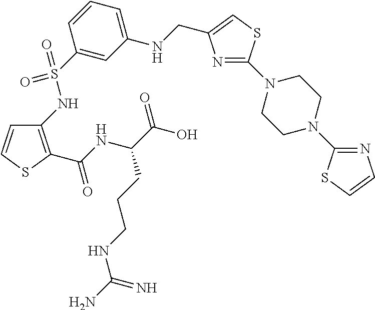 |
| Yield | Green solid, 1.3 mg, 36% |
| LC-MS | Rₜ 5.99 min.; purity 88%; MS m/z - 719 [M + 1]⁺. |
| ID | 77; (S)-5-Guanidino-2-({3-[3-({2-[4-(2-hydroxy-ethyl)- |
| | piperazin-1-yl]-thiazol-4-ylmethyl}-amino)- |
| | benzenesulfonylamino]-thiophene-2-carbonyl}-amino)- |
| | pentanoic acid. |
| Struc- ture | 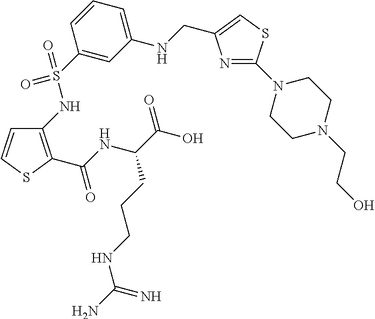 |
| Yield | White solid, 41.3 mg, 61% |
| LC-MS | Rₜ 4.05 min.; purity 99%; MS m/z - 680 [M + 1]⁺. |

### In Vitro Testing for NP-1 Binding and In Vivo Tumour Model Studies

Some of the compounds were tested for NP-1 binding. One compound, 58, was tested for anti-cancer activity in a mouse model bearing xenografts of human lung carcinoma cells. The experimental method and results are shown below.

### General Experimental Methods

### Cell Culture and Adenovirus-Mediated NP-1 Transfection.

Human prostate carcinoma DU145 cells were cultured in growth medium (RPMI 1640 containing 10% FBS and L-glutamine). DU145 cells were seeded at the density of 2×10⁴ cells per well (96-well plates) in 0.1 ml growth medium and transfected with adenovirus vectors containing the full-length open-reading frame of human NP-1. The Ad.NP-1-transfected cells grew for 2 days prior to a binding assay.

### Cell-Based Biotinylated-VEGF-A₁₆₅ Binding.

Confluent Ad.NP-1-transfected cells in 96-well plates were washed twice with phosphate-buffered saline (PBS). The various concentrations of compounds diluted in binding medium (Dulbecco's modified Eagle's medium, 25 mM HEPES pH 7.3 containing 0.1% BSA) were added, followed by addition of 2 nM of bt-VEGF-A₁₆₅. After 2 h of incubation at room temperature, the medium was aspirated and washed three times with PBS. The bound bt-VEGF-A₁₆₅ to NP-1 was detected by streptavidin-horseradish peroxidase conjugates and the enzyme substrate, and measured using a Tecan Genios plate reader at A₄₅₀ nm with a reference wavelength at A₅₉₅ nm. Non-specific binding was determined in the presence of 100-fold excess unlabelled VEGF-A₁₆₅.

### Cell-Free Biotinylated-VEGF-A₁₆₅ Binding.

The 96-well plates were pre-coated with NP1 protein at 3 μg/ml overnight at 4° C. On the following day, the plates were treated with blocking buffer (PBS containing 1% BSA) and washed three times with wash buffer (PBS containing 0.1% Tween-20). The various concentrations of compounds diluted in PBS containing 1% DMSO were added, followed by addition of 0.25 nM of bt-VEGF-A₁₆₅. After 2 h of incubation at room temperature, the plates were washed three times with wash buffer. The bound bt-VEGF-A₁₆₅ to NP-1 was detected by streptavidin-horseradish peroxidase conjugates and the enzyme substrate, and measured using a Tecan Genios plate reader at A₄₅₀ nm with a reference wavelength at A₅₉₅ nm. Nonspecific binding was determined in the absence of NP-1 coated wells of the plates.

The results of the binding studies are shown in Table 11 (below).

TABLE 11

| | Screen | Screen | |
|---|---|---|---|
| Name | (% inhibition at 30 μM) | (% inhibition at 3 μM) | IC₅₀ (μM) |
| 56 | 89 | 57 | |
| 70 | 63 | 25 | |
| 69 | 79 | 37 | |
| 71 | 91 | 53 | |
| 57 | 99 | 98 | 0.040 |
| 58 | 96 | 72 | 0.159 |
| 59 | 94 | 58 | |
| 60 | 96 | 70 | 0.319 |
| 72 | 83 | 31 | |
| 62 | 95 | 68 | |
| 63 | 83 | 49 | |
| 64 | 92 | 72 | |
| 65 | 90 | 40 | |
| 68 | 89 | 37 | |
| 66 | 93 | 71 | |
| 67 | 90 | 44 | |
| 74 | | 55 | |
| 75 | | 63 | |
| 76 | | 68 | |
| 73 | | 69 | |
| 8 | 90 | | 1.8 |
| 9 | 97 | | 3 |
| 10 | 97 | | 2 |
| 11 | 98 | | 1.5 |
| 12 | 96 | | 1.2 |
| 13 | 100 | | 0.591 |
| 14 | 100 | 67 | |
| 15 | 97 | 62 | |
| 16 | 95 | 64 | |
| 17 | 94 | 56 | |
| 18 | 100 | 70 | |
| 20 | 100 | 82 | 2 |
| 22 | 98 | 70 | |
| 23 | 97 | 58 | |
| 24 | 100 | 67 | |
| 25 | 100 | 83 | 0.635 |
| 26 | 100 | 55 | |
| 27 | 97 | 88 | 0.625 |
| 28 | 100 | 96 | 0.216 |
| 29 | 100 | 79 | 0.686 |
| 30 | 100 | 80 | 0.691 |
| 31 | 99 | 86 | 0.105 |
| 33 | 100 | 81 | 0.365 |
| 34 | 94 | 50 | |
| 35 | 96 | 71 | |
| 36 | 98 | 84 | 0.320 |
| 37 | 90 | 68 | 0.637 |
| 38 | 93 | 68 | 0.295 |
| 48 | 99 | 86 | 0.054 |
| 19 | | 56 | |
| 21 | | 73 | |
| 32 | | 75 | |
| 5 | 100 | 92 | 0.014 |
| 1 | | 100 | 0.007 |
| 2 | | 96 | |
| 6 | | 95 | 0.003 |
| 39 | | 61 | |
| 7 | | 94 | 0.005 |
| 3 | | 89 | 0.142 |
| 40 | | 56 | |
| 41 | | 52 | |
| 42 | | 78 | |
| 43 | | 82 | |
| 44 | | 78 | |
| 45 | | 81 | |
| 4 | | 80 | 0.072 |
| 46 | | 65 | |
| 47 | | 86 | 0.130 |
| 49 | | 52 | |
| 50 | | 82 | |
| 51 | | 94 | 0.740 |
| 52 | | 77 | |
| 53 | | 54 | |
| 54 | | 70 | |
| 77 | | 47 | 3.7 |
| 55 | | 71 | 0.525 |

### Lung Cancer Biological Study (In Vivo)

Compound 58 also successfully completed a proof of principle study in a preclinical model of lung cancer. Compound 58 significantly reduced the rate of tumour growth and showed no evidence of toxicity.

In the recent pre-clinical proof of principle study in a murine model of lung cancer, a single daily dose of compound 58 given for two weeks, was shown to reduce the rate of tumour growth by 52% (p=0.017). No evidence of toxicity was seen in the study, consistent with finding of earlier toxicity work at high doses.

### Method

For the efficacy study, human non-small-cell lung carcinoma A549 cells were cultured in growth medium RPMI 1640. The cells at 90% confluence were detached, counted and suspended in PBS to make the final concentration of cells 5×10⁷/ml for inoculation.

### Animal Studies

The compound administration began two weeks after A549 cells were inoculated in female Balb/c nude mice. Compound 58 was dosed intraperitoneally at 80 mg/kg daily for a period of 2 weeks. Tumour volume was monitored by measuring the length and the width of the tumour using an electronic digital caliper (Fisher Scientific) daily for a period of 2 weeks. Tumour volumes were calculated using a formula (length×width²/2). At the end of the experiment, tumours were dissected and weighed.

### Results

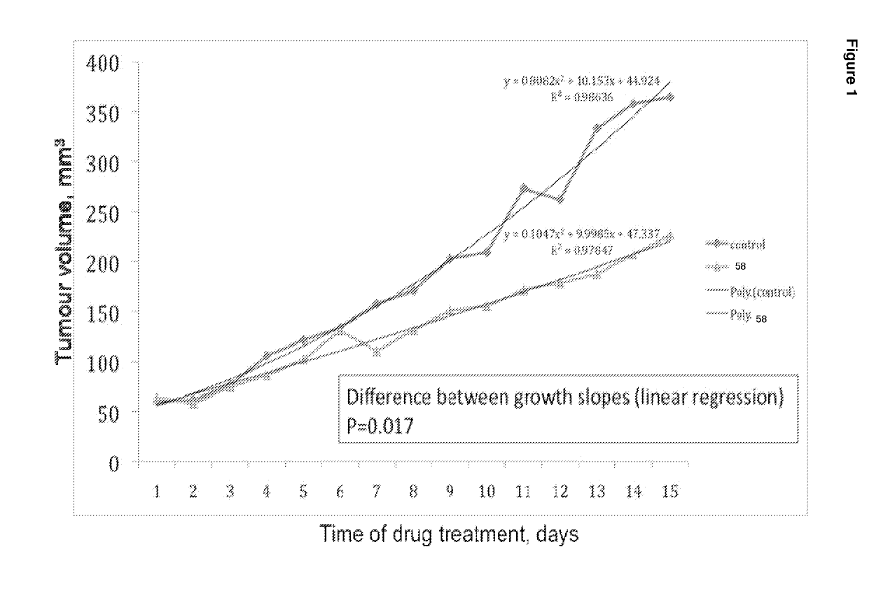

The results of 1n vivo studies are shown in FIG. 1.

The data in FIG. 1 demonstrate that there is a 50% reduction on the tumour growth rate in the compound 58-treated group (p=0.017 by linear regression test). Furthermore, the tumour weight was reduced by 27% (p=0.04 by Mann Whitney test).

1. A compound of formula I: 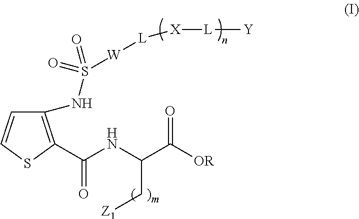
or a pharmaceutically acceptable salt thereof,
wherein:
W is arylene, heteroarylene or 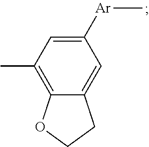
each L is independently alkylene, alkenylene, alkynylene, a direct bond, arylene, cycloalkylene, alkylene-arylene, alkylene-C═O or —C═O;
each X is independently an N-containing heteroarylene, N-containing cycloalkylene or NR;
Y is N-containing heteroaryl, N-containing cycloalkyl, NR₂, OR¹, CN or CO₂R;
Z₁ is 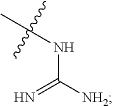
R is H or C₁-C₆ alkyl;
R¹ is H, C₁-C₆ alkyl or an amino acid;
n is 2 to 5; and
m is 1 to 3.

2. A compound according to claim 1, wherein W is arylene.

3. A compound according to formula II: 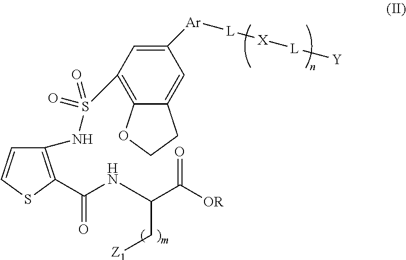
or a pharmaceutically acceptable salt thereof,
wherein:
each L is independently alkylene, alkenylene. alkynylene, a direct bond, arylene, cycloalkylene, alkylene-arylene, or alkylene-C═O;
each X is independently an N-containing heteroarylene, N-containing cycloalkylene or NR;
Y is N-containing heteroaryl, N-containing cycloalkyl, NR, OR¹, CN or CO₂R;
Z₁ is 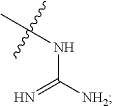
R is H or C₁-C₆ alkyl;
R¹ is H, C₁-C₆ alkyl or an amino acid;
n is 0 to 5; and
m is 1 to 3.

4. A compound according to any preceding claim, wherein at least one L is alkylene

5. A compound according to any preceding claim, wherein at least one L is a direct bond.

6. A compound according to any preceding claim, wherein at least one L is arylene.

7. A compound according to any preceding claim, wherein at least one X is NR.

8. A compound according to any preceding claim, wherein at least one X is a 6-membered cycloalkylene containing at least one N atom.

9. A compound according to any preceding claim, wherein Y is a 6-membered cycloalkyl containing at least one N atom.

10. A compound according to any of claims 1 to 8, wherein Y is a 5-membered heteroaryl containing at least one N atom and preferably one other atom selected from O or S and N.

11. A compound according to any of claims 1 to 8, wherein Y is pyridine.

12. A compound according to any of claims 1 to 8, wherein Y is C₆H₄CN.

13. A compound according to any preceding claim, wherein n is 3, 4 or 5.

14. A compound according to any preceding claim, wherein m is 2 or 3.

15. A compound according to any of claims 1 to 13, wherein m is 1.

16. A compound according to any preceding claim, exemplified and named herein as compound 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76 or 77.

17. A pharmaceutical composition comprising a compound as defined in any preceding claim and a pharmaceutically acceptable excipient.

18. A compound or composition according to any preceding claim, for use in therapy.

19. A compound or composition according to any preceding claim, for use in stimulating nerve repair or in the treatment of neurodegeneration.

20. A compound or composition according to any of claims 1 to 18, for use in the inhibition of platelet aggregation.

21. A compound or composition according to any of claims 1 to 18, for use in the treatment of cancer.

22. A compound or composition according to any of claims 1 to 18, for use in immune system modulation.

23. A compound or composition according to any of claims 1 to 18, for the treatment of HTLVI.

24. A composition comprising a compound according to any of claims 1 to 16 and i) a radionucleus; or ii) a paramagnetic nuclei and a chelate for complexing the paramagnetic nuclei, for use in radioimaging or as a contrast reagent in magnetic resonance imaging.
